(19) United States
(12) Patent Application Publication
(10) Pub. No.: US 2018/0228394 A1
(43) Pub. Date: Aug. 16, 2018

(54) Systems and Methods for Assisting a Subject to Learn to Independently Identify and Alter His or Her Brain State

(71) Applicant: Emtee Associates, L.P., Wilmington, DE (US)

(72) Inventor: Michael Anselme Tansey, Hillsborough, NJ (US)

(73) Assignee: Emtee Associates, L.P., Wilmington, DE (US)

(21) Appl. No.: 15/789,299

(22) Filed: Oct. 20, 2017

Related U.S. Application Data
(63) Continuation-in-part of application No. 14/535,662, filed on Nov. 7, 2014.
Application No. 14/535,662 is a continuation-in-part of application No. 13/373,769, filed on Nov. 30, 2011, now abandoned.
(60) Provisional application No. 61/459,085, filed on Dec. 7, 2010.

(51) Int. Cl. A61B 5/0484; A61B 5/16; G06K 9/00

(52) U.S. Cl. CPC A61B 5/04845; G06K 9/0053; A61B 5/164

(57) ABSTRACT

Systems and methods for assisting a subject to voluntarily alter a brain state. One aspect of the method includes: (i) fitting the subject with equipment to monitor biologic data, the biologic data including a plurality of frequencies in a frequency range; (ii) requesting the subject retain the eyes in a normal open and blinking position for a second time period; (iii) monitoring biologic data during the second time period for the occurrence of a predetermined brain state; (iv) upon the detection of the predetermined brain state, presenting a stimulus to the subject, the stimulus designed to evoke an action performed by the subject; and (v) repeating steps (iii) and (iv).

### CROSS-REFERENCE TO RELATED APPLICATIONS

This application claims the benefit of and is a continuation-in-part of the U.S. non-provisional patent application entitled “Systems and Methods for Detecting Truth Utilizing Biologic Data”, having Ser. No. 14/535,662, filed on Nov. 7, 2014, which is a continuation-in-part of the U.S. non-provisional patent application entitled “Apparatus and Method for Monitoring, Analyzing, and Utilizing Brainwave and Biologic Data at Transition Points Along the Neurochromometric Sequence Whereby a Stimulus Presented to the Central Nervous System Results in Cognition and Volitional Action”, having Ser. No. 13/373,769, filed Nov. 30, 2011, which claims the benefit of the U.S. provisional patent application entitled “Apparatus and Method for Monitoring, Analyzing, and Utilizing Brainwave and Biologic Data at Transition Points Along the Neurochromometric Sequence Whereby a Stimulus Presented to the Central Nervous System Results in Cognition and Volitional Action,” having Ser. No. 61/459,085, filed Dec. 7, 2010, all of which are hereby incorporated by reference in their entireties as if fully set forth herein.

### COPYRIGHT NOTICE

A portion of the disclosure of this patent document contains material which may be subject to copyright protection. The copyright owner has no objection to the facsimile reproduction by anyone of the patent document or the patent disclosure, as it appears in the Patent and Trademark Office patent file or records, but otherwise reserves all copyright whatsoever.

### BACKGROUND OF THE INVENTION

Embodiments of the present invention generally relate to systems and methods for detecting truth and assisting a subject to learn to independently identify and alter his or her brain state. More specifically, the present invention relates to systems and methods for detecting truth and assisting a subject to voluntarily alter a brain state via presentation of a stimulus to a subject upon the detection of a predetermined brain state.

Within the field of neuroscience, it is a commonly accepted view that the brain sets the stage of behavior in a micro-temporal manner. The requisite brain-biologic correlates for cognitive behavior are functionally matched and sorted according to an evolving cognitive-brain archetype in fractions of a second. These micro-state neural networks, along with stimulus refinement and associative response, define and determine cognition. The reflections and correlates of “mind” are also to be found in the interleaved energies of the brain's orchestration of individual functional manifestation. In simpler terms, brain activation networks in the cerebral cortex repeatedly change the state of coordination among its constituent areas on a sub-second time scale to, in an ongoing manner, enable/inform one's conscious awareness and determine one's cognitive state. As such, alterations of brain activation networks yield functional changes in the cognitive state and hence the state of mind of the individual. These pre-cognitive changes in the brainwave activation of regional neural networks can be observed and/or recorded via changes in the specific brainwave activity to determine the individual's brainwave energy signature. The characteristics of the pre-cognitive aspects of one's biologic and brainwave energy signature is what ultimately informs/enables one's flow of consciousness and one's cognitive state.

Cognition is a term which has traditionally been used to refer to one's first person awareness of one's own flow of indwelling consciousness awareness of self and mind. As such one's cognitive state is understood to include diverse mental processes such as un-verbalized and verbalized thinking, conceiving, perceiving, reasoning, one's awareness of the truth and falsehood of information, and self-verbalizations. It also includes any class of mental “behavior” involving symbolizing, insight, expectancy, complex rule usage, intentionality, problem solving, and imagery. When one attends to one's cognitive state/flow of consciousness, one is then amenable to internal acknowledgment, critique, self-assessment as to one's conjectural accuracy and the level of accuracy of one's truthfulness in conveying knowledge and that of data presented by others, and subsequent mental self-talk, prior to any outwardly observable physiological response to it. Digital analysis of the electrical properties of human brainwave activity (cycle-per-second waveforms) provides greater specificity as to the functional correlates of the brain's pre-cognitive, cognitive, somatosensory, and higher order mental function (i.e., the electrophysiological substrate of one's flow of consciousness).

The pre-conscious, pre-cognitive, brain state is different from the conscious, self-aware cognitive state. Pre-cognitive brainwave activation changes that enable a resultant cognitive state are analyzable and identifiable prior to one's conscious awareness of their impact on one's flow of consciousness and cognitive state. As experimentally demonstrated and described by Libet, pre-cognitive changes may take up to one-and-a-half seconds to manifest without any awareness of such changes taking place by the indwelling consciousness of the individual.

Proprioception is the reception of stimuli produced with an organism, which may include one's perception of body awareness. This is a sense that people are frequently not aware of, but rely on continuously, and it is reflected in changes in the person's brainwave activity. The proprioceptive system is often considered to include both the vestibular and kinesthetic systems. Proprioception uses receptors located in the skin, muscles and joints to build a person's sense of his or her body. There are specific nerve receptors for this form of perception termed “proprioreceptors.” These proprioreceptors may sense pressure, light, temperature, sound, and other sensory experiences including, but not limited to: the sense of muscular effort; the sense of force; and the sense of heaviness and relative warmth and position, in limbs and digits, typically sensed in a pre-conscious state. In this, as soon as we are directed by cues internal or external (e.g., verbal) to pay attention to particulars of the above listed senses, these cues may be used to automatically trigger a strong awareness of the cued body sense and a weaker awareness of the sense of other parts of the body and/or lesser mental preoccupation.

Libet replicated the results of Deeke, Grotzinger and Kornhuber, who quantified and isolated Electroencephalographic (“EEG”) brainwave changes enabling what was to be a spontaneous act of individual volition, i.e., flexing a finger. While the people in these studies consciously thought that they were instantly and spontaneously flexing their finger, their brains were observed to be building up the electrical potentials (pre-cognitively) that led to the finger flex for a time period ranging from one (1) second to one-and-a-half (1.5) seconds prior to the avowed spontaneous conscious execution of the finger flex.

When monitoring, analyzing, and utilizing biologic data (e.g., brainwave data, EEG data, electromyographic (“EMG”) data, electrocardiogram (“ECG”) data, galvanic skin response (“GSR”) data, thermal skin temperature change data, and heart rate variability data), there are three key transition points along the path whereby a stimulus presented to the central nervous system results in cognition and volitional action: EEG Stage 1—the pre-stimulus state of activation immediately prior to stimulus introduction; EEG Stage 2—the instant of stimulus presentation which produces an automatic cerebral cascade of related neural network activation; and EEG Stage 3—the elapsed time between the presentation of a stimulus and the subsequent behavioral response. In neuropsychology, EEG Stage 3 is considered to be an index of how fast the thinker can execute the mental operations needed by the task at hand.

Conventional EEG and Quantitative EEG (“qEEG”) methods and apparatus reference EEG energy in wide bands. Wide band (e.g., Delta, Theta, Alpha, and Beta) EEG methodology calculates energy output as follows. Delta is the average of the energy observed in the 0.5 Hz to 1 Hz and each of the 1 Hz through 4 Hz (i.e., 1 Hz, 2 Hz, 3 Hz, and 4 Hz) brainwave bands. Theta is the average of the energy observed in the 4 Hz through 8 Hz brainwave bands. Alpha is the average of the energy observed in the 8 Hz through 12 Hz brainwave bands. Beta is the average of the energy observed in the 13 Hz through 25 Hz brainwave bands. The Delta, Theta, Alpha, and Beta readings are all utilized as independent, standalone measures of EEG activity.

Additionally, wide band activity called Sensorimotor Rhythm (“SMR”) is the average of the energy observed in each of the 12 Hz through 15 Hz brainwave bands, and it is also used as an independent standalone measure of EEG activity. It is known to monitor EEG in terms of the sensed amplitudes and percentages of alpha, theta, beta, delta, and SMR brainwave activity.

Another method EEG analysis includes an event-related-potential (“ERP”). An ERP is the measured brain response that is the direct result of a specific sensory, cognitive, or motor event. More formally, it is any stereotyped electrophysiological response to a stimulus. In some embodiments, the EPR method averages, and thus discards, meaningless, spontaneous EEG waveform fluctuations (e.g., alpha, theta, beta, 1 hz waveforms, etc.) to provide a single, specific outcome measure designated as the P300 wave.

### BRIEF SUMMARY OF THE INVENTION

Briefly stated, in one aspect of the present invention, systems and methods for assisting a subject to learn to independently identify and alter his or her brain state are disclosed. This method includes: (i) fitting the subject with equipment to monitor biologic data, the biologic data including a plurality of frequencies in a frequency range; (ii) requesting the subject retain the eyes in a normal open and blinking position for a second time period; (iii) monitoring biologic data during the second time period for the occurrence of a predetermined brain state; (iv) upon the detection of the predetermined brain state, presenting a stimulus to the subject, the stimulus designed to evoke an action performed by the subject, the action intended to alter the brain state; and (v) repeating steps (iii) and (iv).

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is schematic diagram of a system for monitoring, recording, and/or analyzing biologic data in accordance with one embodiment of the present invention;

FIG. 2 depicts a block diagram of an exemplary computing device for use with the system depicted in FIG. 1;

FIG. 3 depicts an exemplary flowchart for a computer program for obtaining and recording the necessary data for creation of a truth and/or lie scale in accordance with one embodiment of the present invention;

FIG. 4 depicts an exemplary flowchart for a computer program for creating truth or lie scales in accordance with one embodiment of the present invention;

FIG. 5 depicts an exemplary truth scale created in accordance with one embodiment of the present invention;

FIG. 6 depicts an exemplary lie scale created in accordance with one embodiment of the present invention;

FIG. 7 depicts an exemplary charting of baseline EEG biologic data;

FIG. 8 depicts an exemplary charting of EEG biologic data associated with a dominance of mental imagery in general, and intrusive mental imagery in particular, in a brain state in accordance with one embodiment of the present invention;

FIG. 9 depicts an exemplary charting of EEG biologic data associated with a dominance of racing mind in a brain state in accordance with one embodiment of the present invention;

FIG. 10 depicts an exemplary charting of EEG biologic data associated with a dominance of mental fog in a brain state in accordance with one embodiment of the present invention;

FIG. 11 depicts an exemplary charting of EEG biologic data associated with a dominance of a sense of heaviness in limbs, hands, and digits and/or a dominance of perceived muscular tension in a brain state in accordance with one embodiment of the present invention;

FIG. 12 depicts an exemplary charting of EEG biologic data associated with a dominance of a proprioceptive feeling of a part of the body in a brain state in accordance with one embodiment of the present invention;

FIG. 13 depicts an exemplary charting of biologic data associated with a dominance of a proprioceptive feeling of increased or decreased body temperature in a brain state in accordance with one embodiment of the present invention;

FIG. 14 depicts an exemplary brain state chart associated with a first epoch of the onset and/or detection of a predetermined undesirable and/or problematic brain state occurring during a training session in accordance with one embodiment of the present invention;

FIG. 15 depicts an exemplary brain state chart associated with a second epoch that occurs after presentation of a stimulus during a training session in accordance with one embodiment of the present invention;

FIG. 16 depicts an exemplary brain state chart associated with a third epoch that occurs after a second presentation of a stimulus during a training session in accordance with one embodiment of the present invention;

FIG. 17 depicts an exemplary brain state chart associated with a fourth epoch that occurs after a third presentation of a stimulus during a training session in accordance with one embodiment of the present invention;

FIG. 18 depicts an exemplary brain state chart associated with a first epoch of the onset and/or detection of a predetermined undesirable and/or problematic brain state occurring during a training session in accordance with one embodiment of the present invention;

FIG. 19 depicts an exemplary brain state chart associated with a brain state that occurs after a training session in accordance with one embodiment of the present invention; and

FIG. 20 depicts an exemplary process 1300 for obtaining and recording the necessary detecting a brain state, creating baseline data, and/or prompting the presentation of a stimulus to a subject in accordance with one embodiment of the present invention.

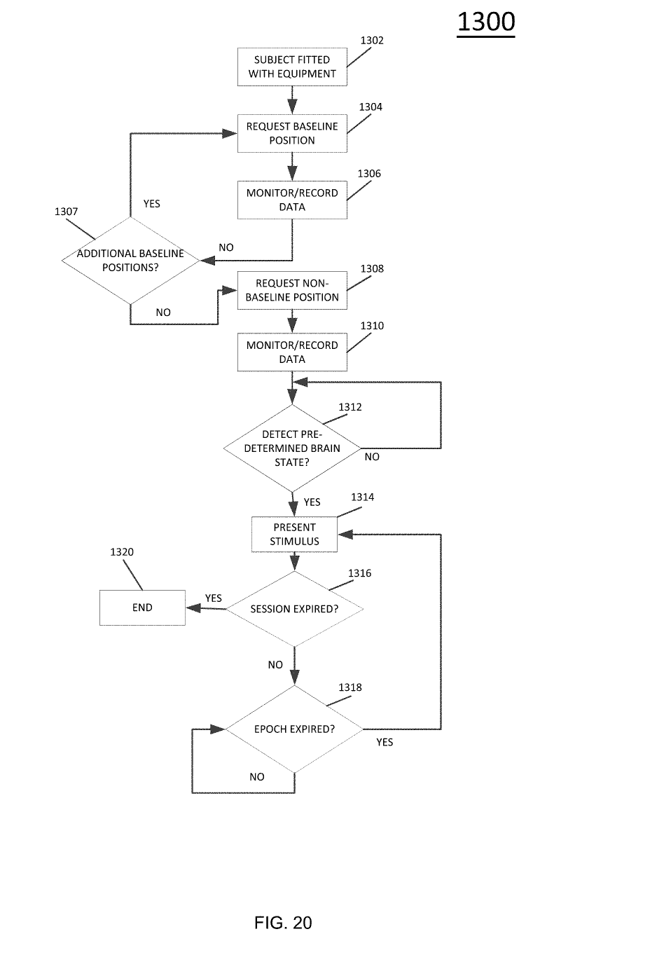

### DETAILED DESCRIPTION OF THE INVENTION

Certain terminology may be used in the following description for convenience only and is not limiting. The words “lower” and “upper” and “top” and “bottom” designate directions in the drawings to which reference is made. The terminology includes the words above specifically mentioned, derivatives thereof and words of similar import.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “an electrode” may include a plurality of electrodes. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

Embodiments of the present invention generally relate to systems and methods for detecting truth utilizing biologic data. More specifically, the present invention relates to systems and methods for detecting truth utilizing biologic data observed during various time periods, or epochs, occurring prior and subsequent to the presentation of a stimulus to a subject and/or prior and subsequent to a volitional action performed by the subject in response to the stimulus. The biologic data collected during these various epochs is analyzed and manipulated to create a truth and/or lie scale for the particular subject. The truth and/or lie scales may then be used to detect the truthfulness of the actions (e.g., answering a question, agreeing to a statement, etc.) performed by the subject associated with the scale(s), wherein the actions are performed in response to a new stimulus (e.g., a statement, a question, etc.).

In one embodiment of the present invention, biologic data is monitored, recorded, analyzed, and/or utilized in three key transition points along the neurochromometric path during a session in which a stimulus is presented to a subject, wherein the stimulus prompts the subject to perform a volitional action. The presentation of the stimulus to the central nervous system of the subject results in cognition and volitional action on the part of the subject. The three key transition points are: 1) the pre-stimulus state of activation immediately prior to stimulus introduction (i.e., EEG Stage 1); 2) the instant of stimulus presentation producing an automatic cerebral cascade of related neural net activation (i.e., EEG Stage 2); and 3) the elapsed time between the presentation of a stimulus and the subsequent behavioral response (i.e., EEG Stage 3). In neuropsychology, EEG Stage 3 is considered to be an index of how fast the thinker can execute the mental operations needed by the task at hand. However, alternate embodiments of the present invention are envisioned in which biologic data observed at any one of these points, or alternate points, along the neurochromometric path may be added, deleted, or substituted without departing from the scope hereof.

Biologic data may be monitored in a variety of ways. In one embodiment of the present, an active electrode is mounted along the midline of the skull of the subject, and reference and ground electrodes are placed on opposite ears of the subject. EEG signals of the subject are detected by the depicted electrode array. However, alternate electrode placement sites may be substituted without departing from the scope of the present invention including, without limitation, any and all of the electrode placement sites identified in the internationally recognized 10-20 or Modified Combinatorial Nomenclature (“MCN”) systems of electrode placement sites. Also, multiple electrode sites may be monitored in lieu of a single site without departing from the scope hereof including, without limitation, ear centric EEG arrays. Also, alternate EEG electrode array embodiments are envisioned in which varying reference and/or ground points and/or locations may be substituted without departing from the scope hereof.

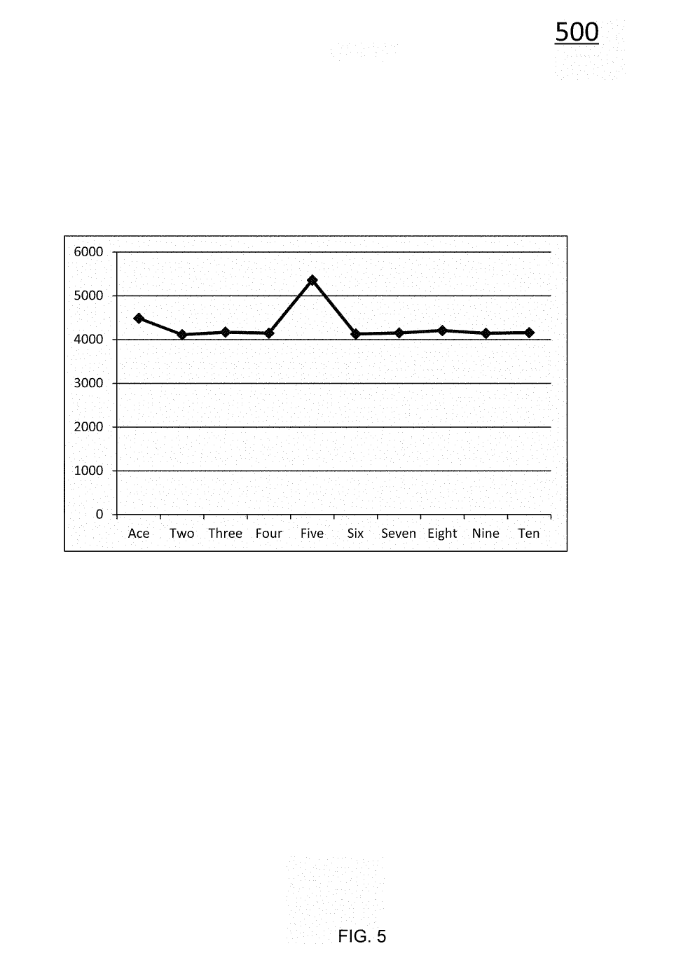

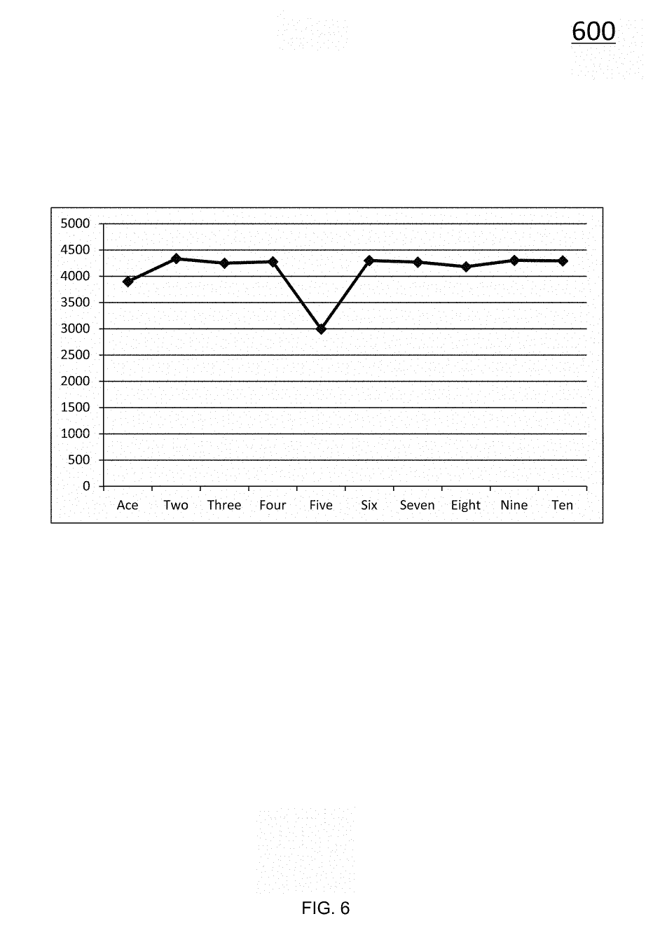

Also, biologic data other than brainwave data may be monitored concurrently with the EEG data via use of additional sensor placements compatible with the type of biologic data to be monitored. Such biologic data may include, but is not limited to, EMG data, ECG data, GSR data, thermal skin temperature change data, and heart rate variability data. Equipment suitable for such measurements is manufactured by companies such as BioRadio. This biologic data may provide additional indication of the physical state of the subject in his or her truth-telling and lie-telling states (in lie detection embodiments) and/or in his or her ability to respond to the stimulus (in brain state identification/training embodiments). This information may be separately graphed or overlaid on the truth and lie detection scales such as those shown in FIGS. 5 and 6. However, such non-brainwave biologic data is not required to implement the present invention.

Further embodiments of the present invention generally relate to assisting a subject to voluntarily alter a brain state for use in, for example, the treatment of a neurological impairment such as Attention Deficit Hyperactivity Disorder (“ADHD”). A brain state herein may include any information indicative of the brainwave signature of, for example, a mind state, body state, proprioceptive state or combination thereof.

The method of treatment includes detection of a particular mind state based upon brainwave data by monitoring brainwave activity for specific mind and/or body changes and providing neurofeedback of such mind-body activity. That is, the biologic data collected during monitored may be analyzed and manipulated in order to identify particular mind and/or body states.

In one aspect of the invention, the method identifies brain wave pairs associated with general brain states of the subject and presents a stimulus to the subject upon the occurrence of certain pre-determined brain states. When the subject is made aware of particular pre-determined brain states, the subject can consciously and/or volitionally alter his or her thinking, and/or proprioceptive sense, to change or attempt to change the subject's current brain state.

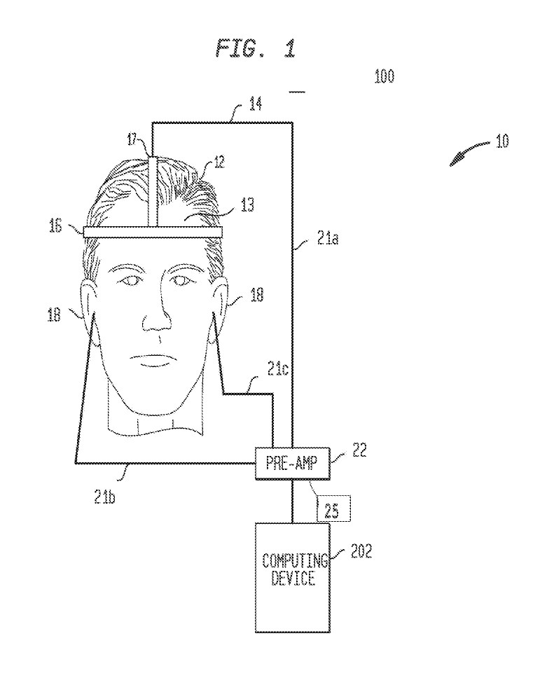

More specifically, referring now to FIG. 1, depicted is a schematic diagram of an exemplary system 10 for monitoring and/or analyzing biologic data in accordance with one embodiment of the present invention. In the depicted embodiment, a subject 12 is monitored with three electrodes 14, 16, and 18. Active electrode 14 is mounted as comfortably as possible along the midline of the skull of subject 12 to overlay the cerebral longitudinal fissure of subject 12. To accommodate this placement, an elasticized headband 15 is placed around the head parallel to the eyebrows and across the middle of the forehead 13 of subject 12. A second elasticized band 17 is placed across the top of the head of subject 12 and attaches to first band 15. Reference electrode 16 is placed on one ear of subject 12, and ground electrode 18 is placed on the other ear of subject 12. In the depicted embodiment, reference and ground electrodes 16 and 18, respectively, are attached with ear clips. However, alternate methods of attachment for electrodes 14, 16, and 18 may be substituted without departing from the scope hereof. It will also be appreciated by those skilled in the art that various sizes, shapes, quantities, and configurations of electrodes 14, 16, and 18 may be substituted without departing from the scope of the present invention. For example, in one alternative embodiment, active, reference, and ground electrodes 14, 16, and 18, respectively, may be incorporated into a skullcap to facilitate the monitoring of brainwaves at any one, any combination of two or more, or the whole array of electrode placement sites defined in the 10-20 or MCN system(s) of electrode sites. Also, hard or soft headbands with or without headphones coupled thereto, headsets, or other devices may be utilized to facilitate monitoring of a plurality of such electrode sites without departing from the scope thereof.

In the depicted embodiment, the signals detected by the electrode system comprised of electrodes 14, 16, and 18 are conveyed by electrode connectors 21 to pre-amplifier 22. However, alternate methods of transmitting these signals may be substituted without departing from the scope hereof including, without limitation, wireless transmission (e.g., Radio Frequency (“RF”), infrared, etc.). Wireless transmission will allow the subject to have a wider range of movement during the monitoring/recording session. In such an embodiment, a remote transmitter may be attached to, for example, first headband 15. The wireless signals may be received by a compatible receiver integral to, or external to, computing device 202.

Preamplifier 22 amplifies and optically isolates the detected bioelectric and EEG brainwave signals received via connectors 21. Preferably preamplifier 22 amplifies these signals by a factor ranging from approximately ten (10) to approximately one hundred (100). In the depicted embodiment, preamplifier 22 is manufactured by Biofeedback Systems as Medical Pre-Amplifier Model PA-2M. However, alternate preamplifiers may be substituted without departing from the scope hereof. Also, although in the depicted embodiment of the present invention, preamplifier 22 is a separate component from computing device 202, it will be appreciated by those skilled in the art that a pre-amplifier capability may be built into computing device 202 rather than using a separate preamplifier 22.

In the depicted embodiment, the bioelectric signals are monitored in a frequency range ranging from approximately zero (0) to approximately one hundred and sixty (160) Hz with bandwidth resolutions of approximately one (1) hertz or less around each frequency in the range. However, alternate embodiments are envisioned in which varying frequency ranges and/or varying bandwidth resolutions are substituted without departing from the scope hereof.

In the depicted embodiment, preamplifier 22 amplifies and optically isolates the detected bioelectric and EEG brainwave signals. The amplified data is transmitted to a signal processor such as computing device 202 as described in greater detail herein. The amplified signals are transmitted to computing device 202 via connection 25 such that they may be read or otherwise interpreted by processing unit 202 of computing device 202. Connection 25 may be any connection capable of transferring signals including, without limitation, wireless connections, USB connections, and the like. The method of detecting EEG is not limited to that disclosed herein. Any suitable apparatus and/or method may be utilized including, without limitation, single channel EEG sensor systems, and ear centric systems.

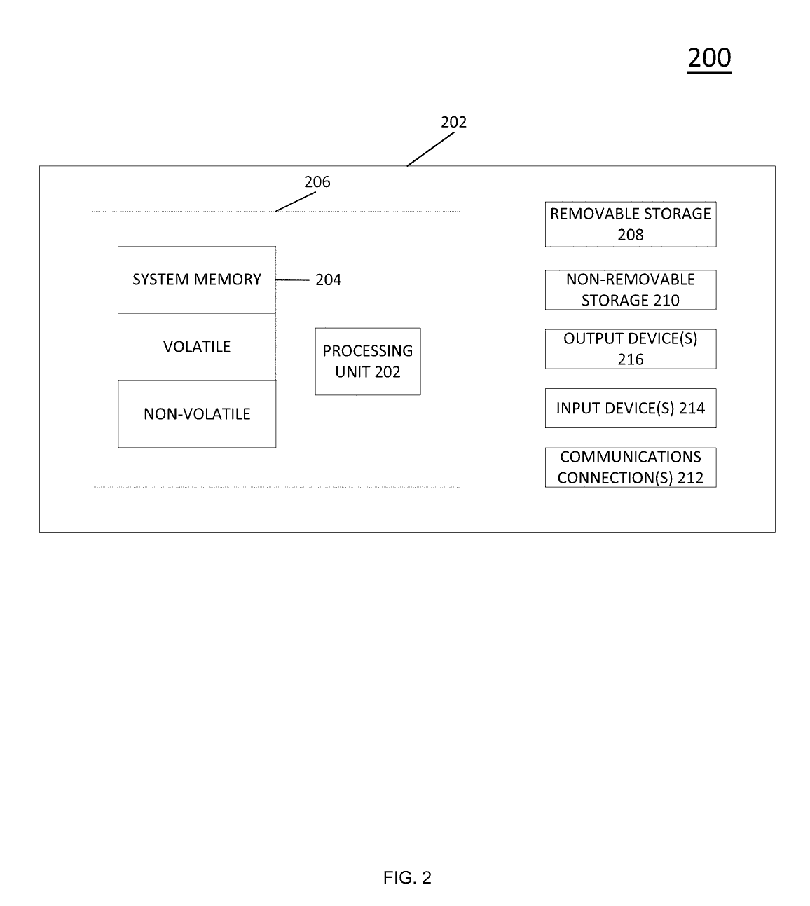

Turning now to FIG. 2, depicted is an exemplary computing device 202 for use with system 10 as described above. The depicted computing device is only one example of a suitable computing device and is not intended to suggest any limitation as to the scope of use or functionality. Numerous other general purpose or special purpose computing system devices, environments, or configurations may be used. Examples of well-known computing systems, environments, and/or configurations that may be suitable for use include, but are not limited to, personal computers (“PCs”), server computers, handheld or laptop devices, multi-processor systems, microprocessor-based systems, network PCs, minicomputers, mainframe computers, cell phones, tablets, embedded systems, distributed computing environments that include any of the above systems or devices, and the like.

Computer-executable instructions such as program modules executed by a computer may be used. Generally, program modules include routines, programs, objects, components, data structures, etc. which perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

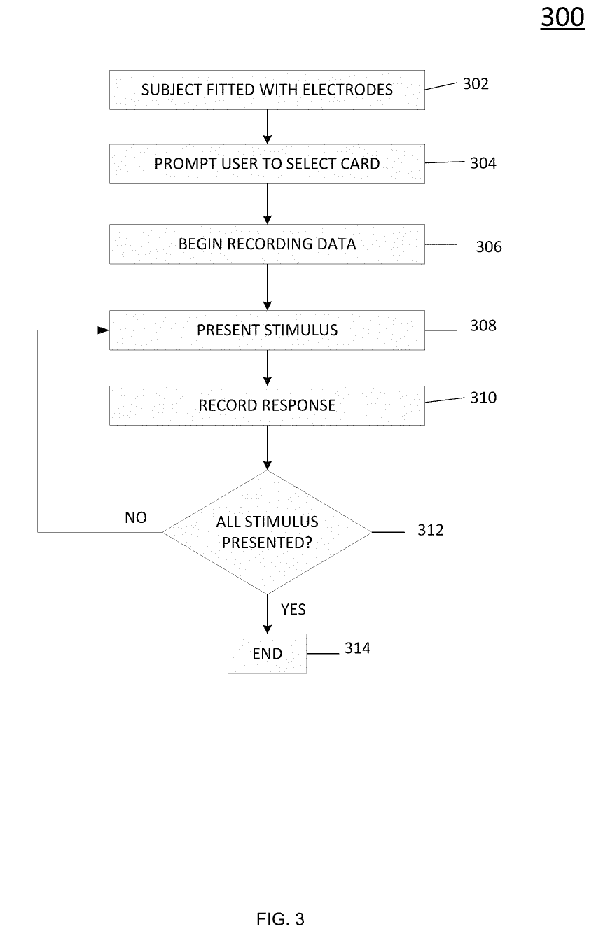

In the depicted embodiment, computing device 202 includes at least one processing unit 202 and at least one memory 204. Depending on the exact configuration and type of the computing device, memory 204 may be volatile (such as random access memory (“RAM”)), non-volatile (such as read-only memory (“ROM”), flash memory, etc.), or some combination of the two. This most basic configuration is illustrated in FIG. 3 by dashed lines 206. In addition to that described herein, computing devices 202 can be any web-enabled handheld device (e.g., cell phone, smart phone, or the like) or personal computer including those operating via Android′, Apple®, and/or Windows® mobile or non-mobile operating systems.

Computing device 202 may have additional features/functionality. For example, computing device 202 may include additional storage (removable and/or non-removable) including, but not limited to, magnetic or optical disks or tape, thumb drives, and external hard drives as applicable. Such additional storage is illustrated in FIG. 3 by removable storage 208 and non-removable storage 310.

Computing device 202 typically includes or is provided with a variety of computer-readable media. Computer-readable media can be any available media that can be accessed by computing device 202 and includes both volatile and non-volatile media, removable and non-removable media. By way of example, and not limitation, computer-readable media may comprise computer storage media and communication media.

Computer storage media includes volatile and non-volatile, removable and non-removable media implemented in any method or technology for storage of information such as computer-readable instructions, data structures, program modules or other data. Memory 204, removable storage 208, and non-removable storage 310 are all examples of computer storage media. Computer storage media includes, but is not limited to, RAM, ROM, electrically erasable programmable read-only memory (“EEPROM”), flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by computing device 202. Any such computer storage media may be part of computing device 202 as applicable.

Computing device 202 may also contain communications connection 312 that allows the device to communicate with other devices including, without limitation, preamplifier 22. Such communications connection 312 is an example of communication media. Communication media typically embodies computer-readable instructions, data structures, program modules and/or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (“RF”), infrared and other wireless media. The term computer-readable media as used herein includes both storage media and communication media.

Computing device 202 may also have input device(s) 314 such as keyboard, mouse, pen, voice input device, touch input device, etc. Output device(s) 316 such as a display, speakers, printer, etc. may also be included. All these devices are generally known to the relevant public and therefore need not be discussed in any detail herein except as provided.

It should be understood that the various techniques described herein may be implemented in connection with hardware or software or, as appropriate, with a combination of both. Thus, the methods and apparatus of the presently disclosed subject matter, or certain aspects or portions thereof, may take the form of program code (i.e., instructions, scripts, and the like) embodied in tangible media, such as floppy diskettes, CD-ROMs, hard drives, flash drives, DVDs or any other machine-readable storage medium wherein, when the program code is loaded into and executed by a machine, such as a computer, the machine becomes an apparatus for practicing the presently disclosed subject matter.

Although exemplary embodiments may refer to utilizing aspects of the presently disclosed subject matter in the context of one or more stand-alone computer systems, the subject matter is not so limited, but rather may be implemented in connection with a multi-computer computing environment. Still further, aspects of the presently disclosed subject matter may be implemented in or across a plurality of processing chips or devices, and storage may similarly be affected across a plurality of computing devices 202. Such devices might include personal computers, network servers, and handheld devices (e.g., cell phones, tablets, smartphones, etc.), for example.

Turning back to FIG. 1, once the amplified signals are received by computing device 202, a fast fourier transform (“FFT”) and/or other bioelectric signal analyses are performed on the collected bioelectric and EEG brainwave signal data. These analyses may be performed via software within the computing device 202 and displayed as desired on the computer screen 28 or stored within the computer or other external data storage device. As discussed above, in the depicted embodiment, the collected bioelectric and EEG brainwave signal data will be in the range of approximately 0 to 190 Hz with a bandwidth resolution of one hertz or less around a given frequency. Raw bioelectric and EEG brainwave signal data and/or its subsequent analysis may be stored in a memory such as memory 204 of computing device 202 or by other means without departing from the scope hereof. However, alternate frequency ranges and/or bandwidth resolutions may be substituted without departing from the scope hereof.

More specifically, computing device 202 performs a FFT on the amplified bioelectric signal to determine the amplitude and/or other characteristics of the bioelectric signal. In the depicted embodiment, the amplitudes of the signals are measured in microvolts. Once these signals are processed, brainwave signatures may be created for the subject. The composite amplitude of the signal is determined by performing an FFT on the amplified signal. A frequency window of up to approximately one (1) Hz around a frequency defines a bioelectric frequency band. Sampling of the bioelectric signals by computing device 202 may be performed, for example, at a rate of about 8,000 samples per second. However, alternate sampling rates and/or bandwidth resolutions may be substituted without departing from the scope hereof.

EEG analysis software allows the user to monitor bioelectric bandwidths of interest from the detected bioelectric signals. A window of 1 hertz or less is the bioelectric bandwidth of interest. Computing device 202 executes EEG analysis software to display particular bandwidths of interest on a computing device output device 316 such as a monitor or the like. In the depicted embodiment, the data is refreshed on the output device 316 in one second intervals or less and the monitoring and processing of the bioelectric signals is ongoing. In the depicted embodiment, EEG analysis software is manufactured by In Sync Institute Inc., as EEG Brainwave Analysis Program, however, alternate software may be substituted without departing from the scope of the present invention. This allows digital readings for each monitored bandwidth's varying peak-to-peak microvolt value to be displayed on output device 316. However, alternate analysis software and/or hardware may be substituted without departing from the scope hereof.

For example, if the user chooses to monitor a bioelectric bandwidth of 14 hertz, the window will be from about 13.5 hertz to about 14.5 hertz. The amplitude values of the bioelectric signals related to the bandwidths of interest can be displayed in a first window of a computer display. Also, an average value of the amplitude of the bioelectric signal over time can be displayed in a second window of the computer display. The amplitude values of the bioelectric signals of the bandwidths of interest collected throughout the entire session are stored in the memory such as memory 204 of the computer such as computing device 202 to allow a monitoring session to be replayed and/or re-analyzed. Also, detected amplitudes for all bandwidths in the 0 to one hundred ninety (190) hertz range can be stored in the memory such as memory 204 of computing device 202.

In the depicted embodiment of the present invention, the data received from electrode connectors 21 by preamplifier 22 is measured and recorded to create truth detection scales (e.g., truth scale and a lie scale) and/or EEG and/or other biologic baseline charts for the subject 12. The biologic data collected and recorded as described above is further evaluated to compare and contrast the signals obtained during EEG Stages 1, 2, and 3 and the changes thereto between these stages. This allows the unconscious and/or pre-cognitive state to be differentiated from the conscious and/or cognitive state of a person's central nervous system when it is responding to a stimulus or other outside influence.

Referring now to FIG. 3, depicted is an exemplary computer program for obtaining the necessary data for creation of a truth detection scale and/or EEG and/or other biologic baseline charts in accordance with the depicted embodiment.

Process 300 starts at 302, at which the subject 12 is fitted with a plurality of electrodes 14, 16, and 18, for example, in the manner discussed above and is seated in front of computing device 202. However, alternate subject locations may be substituted without departed from the scope hereof. One set of stimuli includes a plurality of traditional playing cards having values ranging from one through ten. However, varying stimuli, or sets of stimuli, tailored to specific venues of experimentation and/or questioning such as visual, auditory, or other sensory questioning may be substituted without departing from the scope hereof.

In the depicted embodiment, the stimuli are arranged face down on a table or other surface on which computing device 202 is resting such that they are between the user and the computing device 202. In the depicted embodiment, ten playing cards are used; however, alternate quantities may be substituted without departing from the scope of the present invention. Once the subject is fitted and seated, the subject notifies computing device 202 by clicking a proceed button or the like via input device 314 such as a mouse, touchpad, or the like. However, alternate methods of signaling may be substituted without departing from the scope hereof. Process 300 then proceeds to 302.

At 304, the subject is prompted to select a card by turning the card over via a visual direction displayed to the subject via an output device 316 such as a display device. That is, the subject 12 is provided with a choice and the subject makes a volitional action in response to the choice. Once the subject turns over the desired card such that it is face up and its value can be seen, the subject notifies computing device 202 by clicking a proceed button or the like and process 300 proceeds to 306. For explanation purposes, we will use an example in which the subject turned over a card having a value of five (5).

At 306, process 300 begins recording all data received from preamplifier 22 via execution of EEG analysis software or the like as discussed in greater detail above. Raw data, EEG data, and other biologic data may also be separately recorded or recorded via such software. In the depicted embodiment, the data will continue to be recorded from step 306 until the conclusion of process 300. The data is continuously saved in a memory such as memory 304 for current and future analysis. However, alternate embodiments are envisioned in which recording and/or storing is not a continuous process.

Next, at 308, process 300 presents a stimulus to subject 12. In the depicted embodiment, the first stimulus is the statement “The card that you chose and turned over is an Ace”. At 310, the subject's response is recorded. This response may be entered by the subject by his or her clicking of a “Yes” or “No” button or the like. However, alternate methods of input may be substituted without departing from the scope hereof. In the depicted embodiment, the subject has turned over one card only, for example, a card having a value of five. Prior to the execution of process 300, the subject is directed to answer yes, or to agree with, all of the statements for each card value. Therefore, the subject is not being truthful when agreeing with all statements other than “The card that you chose and turned over is a Five.” In this manner, process 300 is able to record the brainwave activity that occurs when the subject is not telling the truth.

Next, at 312, process 300 determines whether all stimuli have been presented to the user. In the depicted embodiment, the subject is asked if he or she turned over an Ace, Two, Three, Four, Five, Six, Seven, Eight, Nine, and Ten (i.e., ten different stimuli are presented to subject 12). If not all stimuli have been presented, process 300 returns to 308 at which the next stimulus is presented to subject 12. If all of the stimuli have been presented (e.g., all ten statements have been presented and the respective answers recorded), process 300 proceeds to 314, at which it ends and recording of the data received from preamplifier 22 ends. As discussed in greater detail above, from step 306 to step 314, process 300 continuously records the data received from preamplifier 22 for each frequency ranging from approximately 0 Hz (i.e., the start frequency) to approximately one hundred sixty (160) Hz (i.e., the end frequency). The data is recorded in toto in real-time from step 306 through the end of process 300 to allow it to be re-analyzed and/or otherwise used at a future date. Although the depicted embodiment of the present invention utilized ten stimuli, alternate quantities may be substituted without departing from the scope hereof.

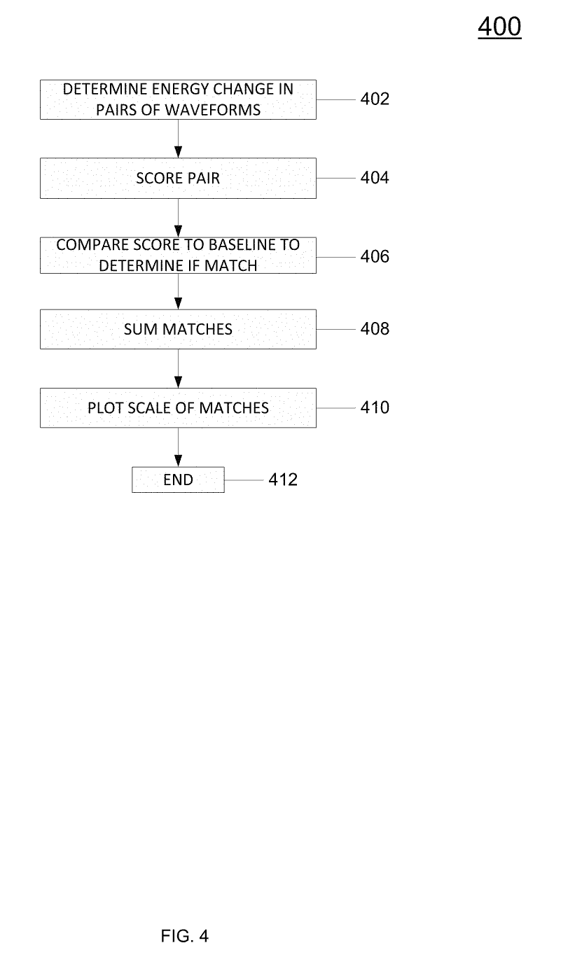

After data for a particular subject 12 has been recorded, truth detection scales may be generated from such data utilizing a process such as process 400 as depicted in FIG. 4. Turning now to FIG. 4, depicted is a flowchart of an exemplary process for generating truth detection scales in accordance with one embodiment of the present invention. Process 400 begins at 402, at which the energy change of each waveform in a pair of waveforms is determined.

In the depicted embodiment, the energy change being measured at step 402 is the change in energy from a second epoch to a third epoch, wherein the second epoch occurs subsequent to presentation of a stimulus and the third epoch occurs subsequent to an action performed by the subject. In the depicted embodiment, the second and third epochs each have a duration of approximately one second. In this manner, the energy change during the waveforms produced in EEG Stage 3 (i.e., the elapsed time between the presentation of a stimulus and the subsequent behavioral response) are monitored. More specifically, in the depicted embodiment, the change in waveform energy is analyzed during the time period that encompasses the action of entering “true” or “false” in response to a stimulus such as that presented at step 308 of process 300. However, alternate epoch timing and/or epoch durations may be substituted without departing from the scope hereof Separate, concurrent analyses for change directionality are computed as additional measures confirming the brain/mental state under consideration. The same spreadsheet or other algorithmic tool may be used; however, the directionality of the data may change. For example, in the depicted embodiment, the directionality of the change in amplitude of any brainwave configuration is monitored and/or recorded including, without limitation: 1) no change in the amplitude of a single one (1) Hz brainwave; 2) a decrease in the amplitude of a single one (1) Hz brainwave; 3) both amplitudes of a selected pair of brainwaves increase; 4) both amplitudes of a selected pair of brainwaves decrease; and/or 5) the amplitudes of a selected pair of brainwaves differ (i.e., one increases and the other decreases). These analyses are merely exemplary and various combinations of brainwaves and/or single brainwaves may be analyzed in a variety of manners without departing from the scope thereof.

In the depicted embodiment, the energy change between a second epoch and a third epoch for a plurality of waveform pairs is analyzed. Data collected or calculated during this time period may be referred to herein as second change data. In the exemplary embodiment shown in FIGS. 1 through 6, a pair set is created for each frequency in the range of frequencies (e.g., 2 Hz through 160 Hz). Each pair set includes a pair for each frequency value in the frequency range other than the frequency value for which the pair set is being created. Each pair includes first pair data and second pair data. The first pair data is second change data for the frequency for which the pair set is being created. The second pair data is second change date for the other frequency associated with the pair.

For example, the pairs are created as follows: the value of each frequency in the monitored range with the exception of 0 and 1 Hz (e.g., 2 Hz through 160 Hz) is paired with all other frequencies in the range. For example, 2 Hz is paired with all frequencies in the range other than itself, 0 Hz, and 1 Hz (i.e., it is paired with 3 Hz through 160 Hz). A pair set may be created for a first frequency (e.g., 2 Hz) as follows. The 2 Hz pair set includes a pair for each frequency value in the frequency range other than the frequency value for which the pair set is being created (3 Hz through 160 Hz). Each pair in the pair set includes first pair data and second pair data. The first pair data is second change data for the frequency for which the pair set is being created (i.e., 2 Hz). The second pair data is second change date for the other frequency associated with the pair (3 Hz through 160 Hz).

That is, the pair set for a first frequency such as 2 Hz includes second change data for a first pair of frequencies 2 Hz and 3 Hz, second change data for a second pair of frequencies 2 Hz and 4 Hz, second change data for a third pair of frequencies 2 Hz and 5 Hz and so on through the final pair of frequencies 2 Hz and 160 Hz. And, similarly, the pair set for 3 Hz pairs second change data for 3 Hz with second change data for all frequencies in the range other than itself, 0 Hz, and 1 Hz (i.e., it is paired with 2 Hz, and 4 Hz through 160 Hz). That is, the pair set for a frequency such as 3 Hz includes second change data for a first pair of frequencies 3 Hz and 2 Hz, second change data for a second pair of frequencies 3 Hz and 4 Hz, second change data for a third pair of frequencies 3 Hz and 5 Hz and so on through the final pair of frequencies 3 Hz and 160 Hz. In this manner, pair sets are created for all frequencies in the range from 2 Hz to 160 Hz. However, alternate frequency ranges may be substituted without departing from the scope of the present invention. Also, alternate frequency pairs and/or pair sets may be substituted without departing from the scope hereof (e.g., all even pairs, all odd pairs, etc.). Once all of the energy change values have been determined for each waveform in all of the pairs of waveforms described above in step 402, process 400 proceeds to 404.

At 404, each pair of waveforms is scored with a pair score. If the energy value for each of both waveforms, of any given pair, change in the desired direction, the pair is given a positive pair score such as a 1. The possible directions for the pair of energy levels are: 1) both energy values change in the same direction; 2) both energy values change in opposite directions; 3) or no change occurs in at least one of the energy values. In the depicted embodiment of the present invention, the desired direction occurs when both energy values in the pair change in the same direction, regardless of the quantity of change. In this scenario, the pair is scored with a positive score value such as a one (1). If the change does not occur in this desired direction, the pair is scored with a negative pair score such as a zero (0). However, alternate embodiments are envisioned in which an analysis may be performed for different desired directions without departing from the scope hereof.

Next, at step 406, the pair scored in step 406 is compared to a baseline value to determine whether there is a match. In the depicted embodiment, each frequency in the range of frequencies has one (1) baseline value for each stimulus presented to the subject such as, for example, during step 308 of process 300. The baseline value is a one (1) or a zero (0) and is created in a manner similar to the scoring of the pairs, however, the baseline value is created based upon brainwave activity measured between the first and second epochs.

More specifically, in the depicted embodiment, the energy change utilized to create the baseline for each frequency is the change in energy from a first epoch to a second epoch, wherein the first epoch occurs during presentation of a stimulus (i.e., the pre-conscious sensory detection phase) and the second epoch occurs subsequent to presentation of a stimulus (i.e., the perception/comprehension phase) (the same second epoch as described above with respect to step 402). Data collected or calculated during this time period may be referred to herein as first change data. In the depicted embodiment, the first and second epochs each have a duration of approximately one (1) second. In this manner, the energy change during the waveforms produced in EEG Stage 2 (i.e., the instant of stimulus presentation which produces an automatic cerebral cascade of related neural network activation)) is utilized to create a baseline value. More specifically, in the depicted embodiment, the change in waveform energy is analyzed during the time period that encompasses the presentation of the stimulus (such as the statement presented at step 308 of process 300) to the subject and the subject's cognition of the stimulus (e.g., statement). However, alternate epoch timing and/or epoch durations may be substituted for creation of a baseline without departing from the scope hereof.

These changes in energy levels for each of the baseline pairs of waveforms (i.e., the same pairs discussed above with respect to step 402 except that the data is recorded between the first and second epochs instead of the second and third epochs) are scored with a baseline pair score in a similar manner as discussed above.

More specifically, each pair of baseline waveforms is scored with a baseline pair score. If the energy value for each of both waveforms, of any given baseline pair, change in the desired direction, the baseline pair is given a positive baseline pair score such as a 1. The possible directions for the baseline pair of energy levels are: 1) both energy values change in the same direction; 2) both energy values change in opposite directions; 3) or no change occurs in at least one of the energy values. In the depicted embodiment of the present invention, the desired direction occurs when both energy values in the baseline pair change in the same direction, regardless of the quantity of change. In this scenario, the baseline pair is scored as a positive baseline score such as a one (1). If the change does not occur in this desired direction, the baseline pair is scored with a negative pair score such as a zero (0). However, alternate embodiments are envisioned in which an analysis may be performed for different desired directions without departing from the scope hereof.

For each frequency and/or each stimulus, a first baseline value is assigned to a frequency of the frequency range and/or a stimulus for which a quantity of positive baseline pair scores is equal to or exceeds a predetermine percentage. For example, if sixty percent (60%) or more of the scored baseline pairs associated with the specific frequency and stimulus have been scored with a one, then the baseline value is set to a first baseline value (e.g., a one). Otherwise, the baseline value is set to a second baseline value (e.g., zero). However, alternate predetermined percentages other than sixty percent (60%) may be substituted without departing from the scope hereof. Also, a cutoff value may be used in lieu of a predetermined percentage without departing from the scope hereof.

Therefore, for example, the baseline value for a frequency of 2 Hz for a stimulus in which the subject agreed with the statement “The card that you chose and turned over is an Ace” is determined by scoring the baseline energy change for all 2 Hz frequency pairs in the baseline pair set associated with that frequency and/or stimulus (i.e., 2 Hz and 3 Hz, 2 Hz and 4 Hz, 2 Hz and 5 Hz and so on through 2 Hz and 160 Hz). If sixty (60) percent or more of these 2 HZ pairs have a positive baseline pair score of, for example, one, then the baseline value for that stimulus and 2 Hz is set to a first baseline value (e.g., one). If less than sixty (60) percent of these pairs have a positive baseline pair score of, for example, one, then the baseline value for that stimulus and 2 Hz is set to a second baseline value (e.g., zero).

At step 408, each pair scored at step 406 in the pair set for a particular frequency and/or a particular stimulus is compared to the baseline value associated with the particular frequency and/or stimulus to determine if there is a match between the pair's score and the baseline value. Each pair that has a score that matches the baseline value is assigned a match value (e.g., one) and each pair that has a score that does not match the baseline value is assigned a non-match value (e.g., zero). The match values are then summed for the specific frequency and/or stimulus. This process is performed for each frequency per stimulus. Process 400 then proceeds to 410.

At 410, the quantity of matches for each stimulus is summed to create a total match value. For example, for the stimulus in which the subject was asked if he or she turned over an ace, the quantity of matches for 2 Hz is summed with the quantity of matches for 3 Hz, 4 Hz, and 5 Hz through 76 Hz. The same summing process is performed for each of the stimuli presented to the subject such as, for example, at step 308 of process 300. These sums may be utilized to create a truth scale as discussed with respect to step 412.

Alternatively, rather than assigning a value of, for example, one to matches, this value may be assigned to non-matches as a non-match value. In this scenario, the quantity of non-match values for each stimulus may also be summed. For example, for the stimulus in which the subject was asked if he or she turned over an ace, the quantity of non-matches for 2 Hz is summed with the quantity of non-matches for 3 Hz, 4 Hz, and 5 Hz through 76 Hz. These values may be utilized to create a lie scale as depicted in FIG. 6.

Next, at step 412, the total match values determined at step 410 are plotted to create a truth scale. One such exemplary truth scale is depicted in FIG. 5. The stimuli are plotted on the y axis and the quantity of matches associated with each stimulus is plotted on the x axis. As can be seen in the exemplary scale shown in FIG. 5, the total match quantity for the one stimulus in which the subject was truthfully in agreement with the statement is significantly higher than the quantity of matches for the stimuli for which a false agreement was provided. In the depicted embodiment, the difference is approximately one thousand, however, alternate values may be determined when performing the processes described herein without departing from the scope of the present invention.

Alternatively, at step 412, the sums of non-matches determined at step 410 may be plotted to create a lie scale. One such exemplary truth scale is depicted in FIG. 6. The stimuli are plotted on the y axis and the quantity of non-matches associated with each stimulus is plotted on the x axis. As can be seen in the exemplary scale shown in FIG. 6, the quantity of non-matches for the one stimulus in which the subject was truthfully in agreement with the statement is significantly lower than the quantity of non-matches for the stimuli for which false agreement was provided. In the depicted embodiment, the difference is slightly less than one thousand, however, alternate values may be determined when performing the processes described herein without departing from the scope of the present invention.

Once a truth or lie scale has been generated in accordance with the above processes, one or both of these scales may be used to determine whether a subject is being truthful when responding to a new stimulus (e.g., a statement or a question). The new stimulus is presented in the same manner as described above with respect to steps 306 through 314 of process 300 and the biologic data is recorded during the time period prior to, during, and subsequent to presentation of the stimulus and the subject providing an action in response thereto.

After the biologic data has been recorded, it may be analyzed in a manner such as that described above with respect to FIG. 4. When all of the matches have been summed such as described above with respect to step 408 of FIG. 4, the sum may be compared to the values on the truth or lie scale to determine in which range the sum falls. For example, if utilizing the truth scale depicted in FIG. 5, a sum equal to or greater than four thousand seven hundred fifty (4,750) may be deemed to be a truthful response and a sum less than four thousand seven hundred fifty may be deemed to be a non-truthful response. Or, if a lie scale is utilized such as that depicted in FIG. 6, a sum equal to or less than three thousand five hundred (3,500) may be deemed to be a truthful response and a sum greater than three thousand five hundred may be deemed to be a non-truthful response. However, other cut-off values may be utilized to determine truthfulness or non-truthfulness without departing from the scope of the present invention.

Other embodiments of the present invention generally relate to systems and methods for assisting a subject to learn to independently identify and alter his or her brain state. In one aspect, such methods may be utilized for the treatment of neurological impairments (e.g., ADHD).

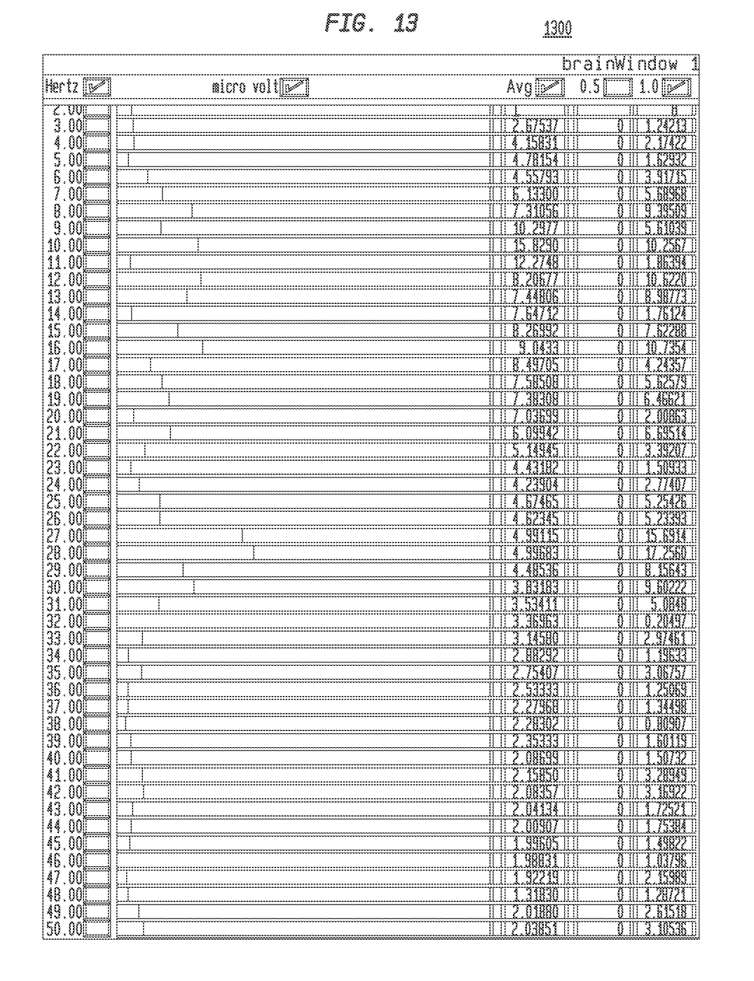

Referring now to FIG. 13, depicted is an exemplary flowchart of a process 1300 for obtaining the necessary data for detecting a brain state and presenting a stimulus to a subject in accordance with the depicted embodiment. A process such as process 1300 may be utilized, for example, to establish a pre-training baseline and/or targeted training data that includes subject specific EEG signatures of, for example, various mind state, body states, proprioceptive states or combinations thereof as evidence, experienced, and prompted for, during the baseline session including, without limitation: mindfulness; clarity of mind; mental fog; racing mind; calm mind; “heaviness” in arms, hands, and fingers; and a sense of surface temperature on arms, hands, and fingers. Via such a method, biologic and EEG brainwave data may be utilized to identify and/or chart brain states and to train increases or decreases in pre-identified brain states, enabling training of a subject to independently identify and alter his or her brain state. In one scenario, this altering may result in diminishing surges in specific brainwaves enabling a particular brain state.

Process 1300 starts at 1302, at which a subject 12 is fitted with a plurality of electrodes 14, 16, and 18, for example, in the manner discussed above. The subject may be seated in front of a computing device such as computing device 202 or may be in another suitable location, if a computing device 202 shall be utilized for presentation of a stimulus to the subject. Such stimulus may include, but is not limited to, a visual stimulus (e.g., an image, a change in color of a display of a computing device, etc.), a vibratory stimulus, and/or an auditory stimulus such as a voice, a tone, or recorded music.

In some embodiments of the invention, the stimulus is a predetermined cue such as a verbal cue. For example, the verbal cue may be the spoken words “calm mind”, however, the invention is not so limited. A wide variety of stimuli may be generated to maximize the ability of the subject to identify his or her brain state and act to alter same.

In one embodiment of the present invention, an interactive feedback mode is included. In this mode, the volume of the auditory stimulus increases during a change from a first non-preferred brain state to a second preferred brain state. Or, alternatively, the volume of the auditory stimulus may decrease during a change from a first non-preferred brain state to a second preferred brain state. Or alternatively, the volume of the auditory stimulus increases (or decreases) during an increase (or decrease) in strength of the frequencies associated with a preferred or non-preferred brain state.

If a device such as headphones will be utilized for presentation of the stimulus, such a device is also fitted to the subject during step 1300. Although the stimulus may be presented by a computing device such as computing device 202, such a device is not required. The stimulus may be presented via any suitable method.

Once the subject is fitted, he or she may notify a computing device 202 of same by clicking a proceed button or the like via an input device 314 such as a mouse, touchpad, or the like. Alternatively, a subject may verbally notify a provider of the method that he or she is fitted. Process 1300 then proceeds to 1304.

At 1304, the subject is prompted to assume a baseline analysis position. In one aspect of the invention, this position is one in which the subject is seated, substantially non-moving, with eyes in a closed position. However, alternate positions may be substituted. Once the subject is in the baseline position, process 1300 proceeds to 1306.

At 1306, process 1300 begins recording all data received from preamplifier 22 via execution of EEG analysis software or the like as discussed in greater detail above. Raw data, EEG data, and/or other biologic data may also be separately recorded or recorded via such software. In the depicted embodiment, the data will be recorded when the time period for collection of baseline data expires. One exemplary time period is two (2) minutes, however, the invention is not so limited. The baseline data is saved in a memory such as memory 304 for current and future analysis. Once the baseline data has been recorded, individualized baseline EEG brainwave and/or brain state charts may be generated depicting the energy values and/or changes of each waveform in the range of monitored waveforms. Then, process 1300 proceeds to 1307.

At 1307, process 1300 queries whether another baseline position requires measurement. If yes, process 1300 returns to 1304, at which the subject assumes the position for the new baseline to be recorded. For example, if a baseline position for muscular tension is to be recorded, the subject may be asked to be aware of the proprioceptive sense and/or enabling brain state comprising “warm hands”. Once the subject is in the proper position and/or state of mind, process 1300 proceeds to 1306 at which the raw data, EEG data, and/or other biologic data may be separately recorded or recorded via such software. Once the new baseline data has been recorded, individualized baseline EEG brainwave and/or brain state charts may be generated depicting the energy values and/or changes of each waveform in the range of monitored waveforms. And, process 1300 proceeds to 1307.

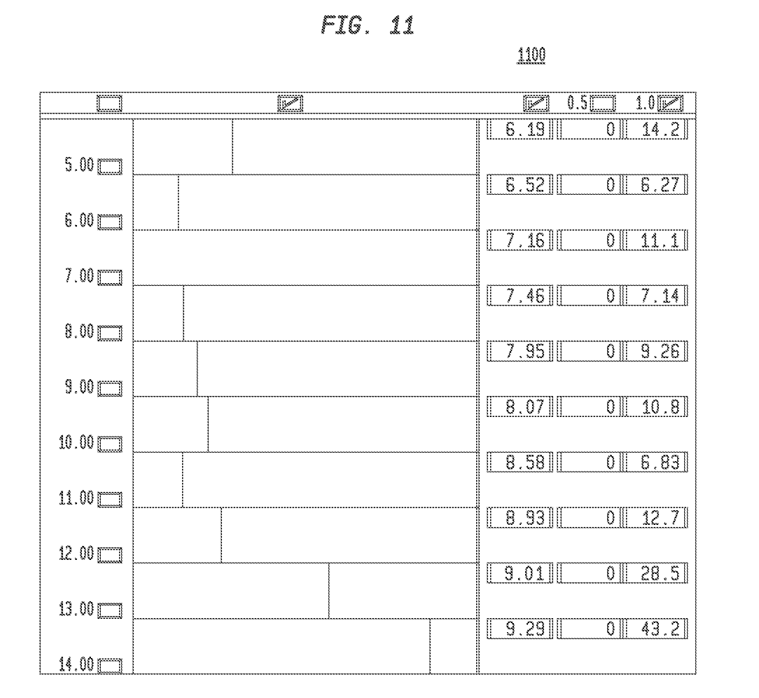

Alternatively, if at 1306, no additional baseline positions require recording, process 1300 proceeds to 1308. Once overall baseline EEG brainwave characteristics including personal EEG charting of various proprioceptive sense states and/or enabling brain states (e.g., mindfulness, clarity of mind, mental fog, racing mind, calm mind, “heaviness” in arms, hands, and fingers, and a sense of surface temperature on arms, hands, and fingers) prompted for during the baseline session are established, volitional training may begin using whatever effective cues were identified in the baseline session to be able to effect the desired awareness and shift desired. FIGS. 11 to 14 are exemplary training screens/brain state charts that include ten (10) brainwave bands and that are used to enable a subject to be able to independently (i.e., of their own volition) become aware of, and then alter a post-baseline identified proprioceptive and/or brain state of interest. The composition of training screens and brain state charts, and the method of use thereof, may be tailored to the individual as needed.

At 1308, the subject is prompted to assume the non-baseline analysis position. In one aspect of the invention, this position is one in which the subject is seated, substantially non-moving, with eyes in a normal open and blinking position. However, alternate positions may be substituted. Once the subject is in the non-baseline position, process 1300 proceeds to 1310.

At 1310, process 1300 begins recording all data received from preamplifier 22 via execution of EEG analysis software or the like as discussed in greater detail above. Raw data may also be separately recorded or recorded via such software. In the depicted embodiment, the data will continue to be recorded from step 1310 until the conclusion of process 1300. The data is continuously saved in a memory such as memory 304 for current and future analysis. However, alternate embodiments are envisioned in which recording and/or storing is not a continuous process. The process may conclude at any time as determined by the subject. Or, in an alternate embodiment, the process concludes upon the expiration of a predetermined time period. One exemplary time period is twenty (20) minutes, however, the invention is not so limited.

Next, at 1312, process 1300 queries the monitored data to determine whether a pre-selected brain state or brain states have been identified. For example, when a process such as process 1300 is utilized to detect ADHD, the pre-selected brain state(s) may be Racing Mind. If the pre-selected brain state(s) are not detected, process 1300 continues to monitor the monitored and/or recorded data for the pre-selected brain state(s). If the pre-selected brain state(s) are detected, process 1300 proceeds to 1314. The subject is in a pre-cognition state in which the subject is experiencing a specific, predefined brain state, but the subject is not necessarily aware of his or her current brain state.

At 1314, process 1300 presents the pre-determined stimulus to subject 12 and the subject is now in a state of cognition. Upon being presented with the stimulus, the subject will actively attempt to alter his or her brain state by controlling his or her thoughts and is therefore in a state of volitional action. For example, in the case of a detected Racing Mind state, the subject will actively attempt to calm his or her mind via using a pre-determined personal cue that effectively slows and clears the subject's thought process, for example, but not limited to, being asked to recall a pleasant memory, favorite place, or the like.

After presentation of the stimulus, process 1300 proceeds to 1316, at which process 1300 queries to determine whether the session has expired. For example, a session may be thirty (30) minutes or the like, and all monitoring and stimulus might cease at the end of the session. However, alternate time periods may be substituted and/or multiple sessions could be performed simultaneously. If the session has expired, process 1300 proceeds to 1320, at which it ends.

If, at 1316, the session has not expired, process 1300 proceeds to 1318, at which process 1300 determines whether the epoch has expired. If no, process 1300 remains at 1318, until the epoch expires, at which point it returns to 1314, at which the stimulus is presented again. In this manner, the stimulus is continually re-presented until the session ends at 1320. At such point, the recording of the data received from preamplifier 22 ends.

In the depicted embodiment, epochs have a duration of one (1) second and there is no time between the epochs. However, alternate epoch durations and timing between epochs may be substituted without departing from the scope hereof. As discussed in greater detail above, from step 1310 to step 1320, process 1300 continuously records the data received from preamplifier 22 for each frequency in the measured frequency range. In the depicted embodiment, the frequency range ranges from approximately two (2) Hz (i.e., the start frequency) to approximately one hundred ninety (190) Hz (i.e., the end frequency). The data is recorded in toto in real-time from step 1310 through the end of process 1300 to allow it to be re-analyzed and/or otherwise used at a future date. Although the depicted embodiment of the present invention measures frequencies ranging from two hertz to one hundred ninety hertz, alternate frequency ranges may be substituted without departing from the scope hereof. Also, alternate embodiments are envisioned in which, after a predetermined number of stimuli are presented (e.g., one, two, three, etc.), process 1300 returns to 1312 at which it continues to monitor for the re-occurrence of the pre-selected brain state(s) until a point at which the session ends.

Through a process such as process 1300 and repeated sessions of application of process 1300, the subject eventually learns how to acknowledge and/or self identify the occurrence of an undesirable brain state and volitionally take action to shift the subject's brain state to a preferred brain state.

In a further embodiment of the present invention, the monitored and/or recorded EEG and other biologic data provides tracking data for the time line of the occurrence of brain states relative to volitional actions taken by the subject.

In some aspects of the invention, the brain state is defined by co-varying brainwave bands or paired brainwave activity. The change in the brain state may be indicated by a surge in a particular brainwave pair. A surge may be quantified as two times as much energy as any of the rest of the brainwaves measured, however, alternate surge quantifications may be substituted without departing from the scope of the present invention.

The recorded brainwave data may also be analyzed to identify any one or more of the following four change states: a first change state in which there is an increase in amplitude in excess of a predetermined percentage for any one of the frequencies in the frequency range; a second change state in which there is a decrease in amplitude in excess of a predetermined percentage for any one of the frequencies in the frequency range; a third change state in which there is an increase in amplitude in excess of a predetermined percentage for both of a pair of the frequencies in the frequency range; a fourth change state in which there is a decrease in amplitude in excess of a predetermined percentage for both of a pair of frequencies in the frequency range; and a frequency of one of a predetermined pair of frequencies increases and the other frequency of the predetermined pair of frequencies decreases. These possible change states depicting the change in the energy states of the predetermined pairs of brainwaves may indicate, for example, the negative influence of intrusive thoughts, partial attention, intrusive feelings and imagery, which may then be addressed via pre-determined tailored changes in the instruction set.

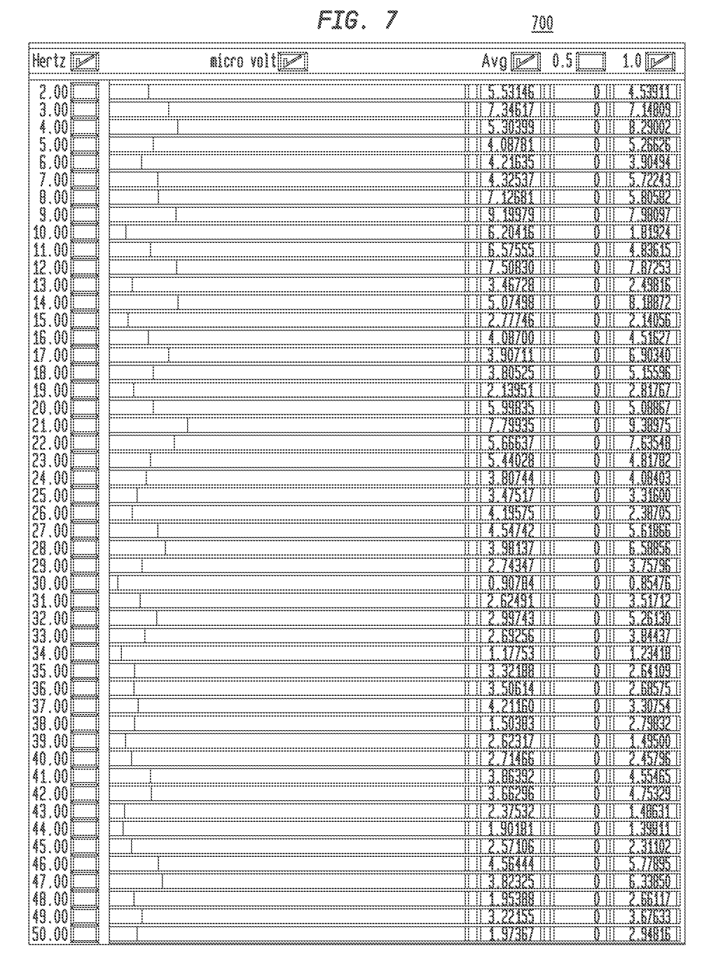

FIGS. 7-12 depict brainwave patterns that may be generated by a subject during an exemplary method of treatment for a neurological impairment or other use of the systems and methods discussed herein. The depicted brainwave patterns correspond to specific brain states. Monitored and/or recorded data may be matched to known EEG signatures where there is a clearly large preponderance of EEG energy in any given pair of brainwaves enabling, for that subject, a recurring brain-wave signature that informs powerfully, whether positively or negatively.

FIG. 7 depicts an exemplary baseline brain state chart 700 illustrating the baseline measurement of brainwaves for a particular subject. In many cases, baseline data may be inherently un-remarkable, as in FIG. 7, with no clearly large preponderance of EEG energy in any given pair of brainwaves indicating the subject's faculties of consciousness, thought, mindfulness, and body awareness. That is, there are no wavelengths surging, i.e., no one pair of frequencies shows a significant difference from the remaining frequencies.

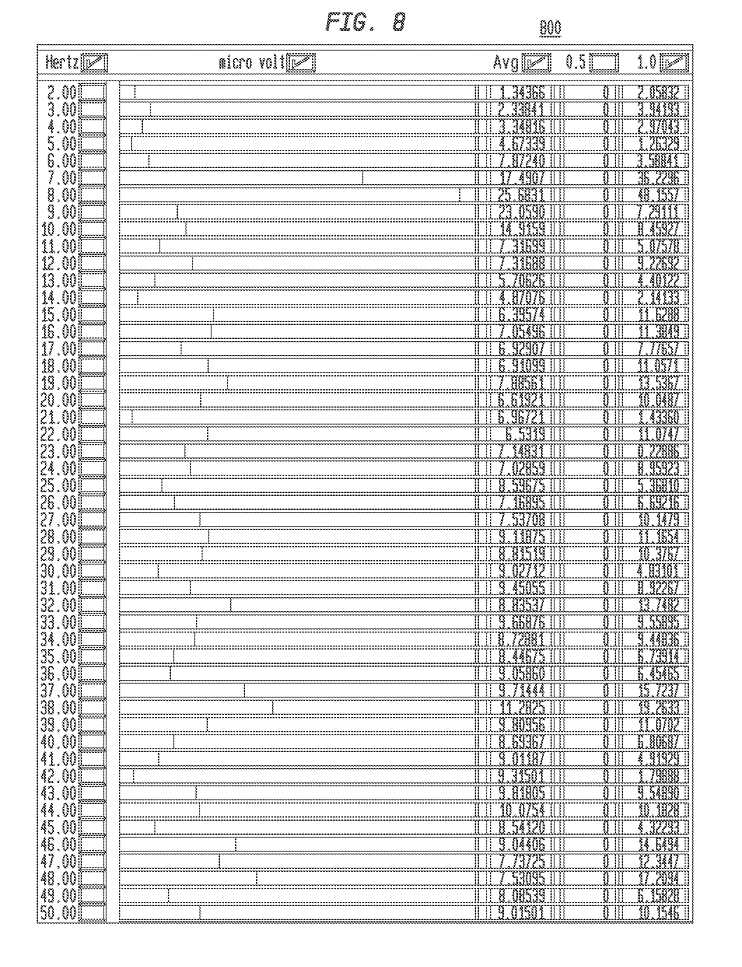

One such strong consciousness informing brain-mind-body baseline is presented in FIG. 8 in which the 7 Hz and 8 Hz brainwaves co-vary with a clearly large preponderance of EEG energy across the EEG spectrum indicating strong mental imagery within the subject's indwelling consciousness. FIG. 8 shows a surge in brainwaves in the brain state chart 800 at the brainwave pair 7 Hz and 8 Hz. The surge in this particular pair of brainwaves typifies mental imagery such as mental visualization and intrusive mental images.

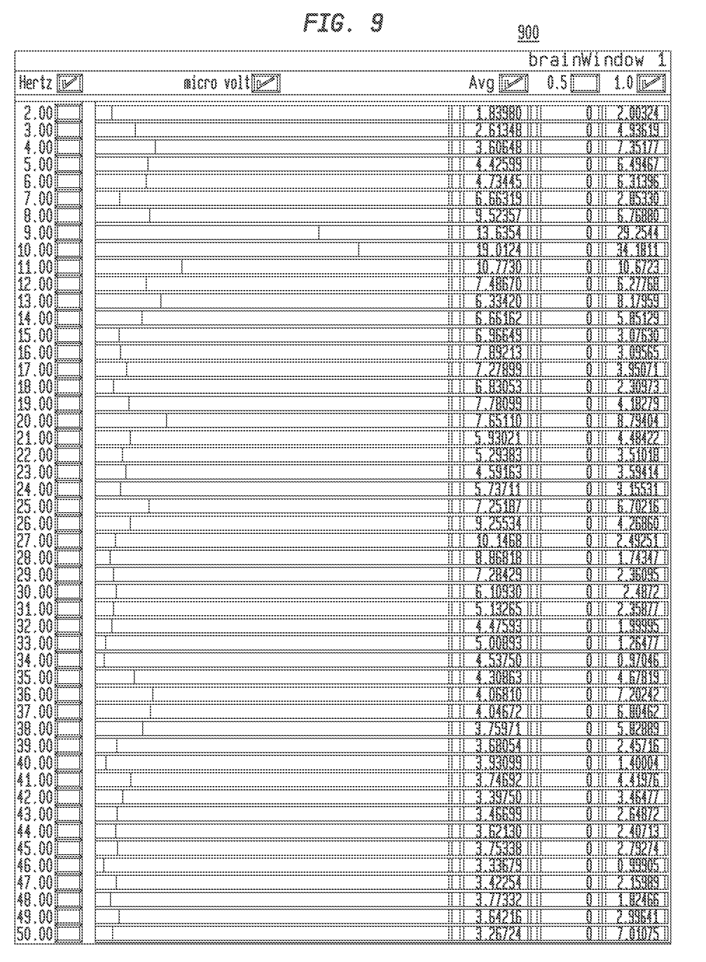

Yet another such strong consciousness informing brain-mind-body baseline is presented in FIG. 9, in which the 9 Hz and 10 Hz brainwaves co-vary with a clearly large preponderance of EEG energy across the EEG spectrum indicating racing mind within the subject's consciousness. FIG. 9 demonstrates a surge in the brain state chart 900 at the brainwave pair 9 Hz and 10 Hz. The surge in this particular pair of brainwaves is typical of a “Racing Mind,” a brain state typical of a subject diagnosed with ADHD and/or a bi-polar disorder. Racing mind may also include obsessive internal self-talk.

In some scenarios, brain state charts such as those depicted herein, may depict a change in amplitude event from the preceding epoch. In FIG. 9, the amplitudes of 9 Hz and 10 Hz increased from the prior epoch in a manner in which their amplitudes are clearly much higher than the amplitudes of all of the other frequencies in EEG spectrum sampled. Upon detection of such a pre-identified, dominating EEG event, a pre-selected and programmed cue may be given to help the subject voluntarily shift to a preferred brain state such as, for example, the brain state exemplified in FIG. 13 for a dominance of awareness of skin surface temperature (i.e. such as “warm hands”). If successful, the overarching dominance of the 9 Hz and 10 Hz amplitudes would decrease suddenly and a sudden surge of the 27 Hz and 28 Hz amplitudes would occur. In this manner, when brain states are identified, the subject may be taught how to control the brain states and/or to swap non-preferred brain states for preferred brain states.

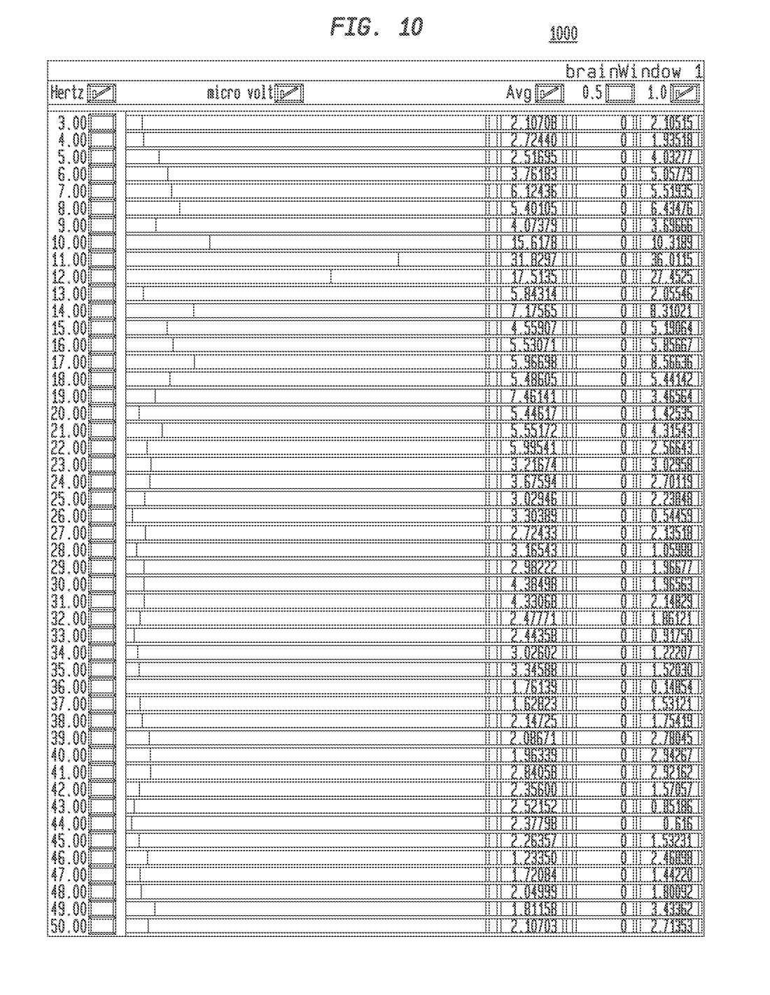

A further such strong consciousness informing brain-mind-body baseline is presented in FIG. 10, in which the 11 Hz and 12 Hz brainwaves co-vary with a clearly large preponderance of EEG energy across the EEG spectrum indicating the level of mental fog within the subject's indwelling consciousness. FIG. 10, showing a surge in the brain state chart 1000 at the brainwave pair 11 Hz and 12 Hz, may be characterized as indicative of the subject's level of mental fog, or mental zoning-out/mental mindfulness.

FIG. 11 shows a brain state chart 1100 characterized as a brain state enabling the release of muscular tension as well as general state of muscular calmness. The brainwave pair, 13 Hz and 14 Hz are surging.

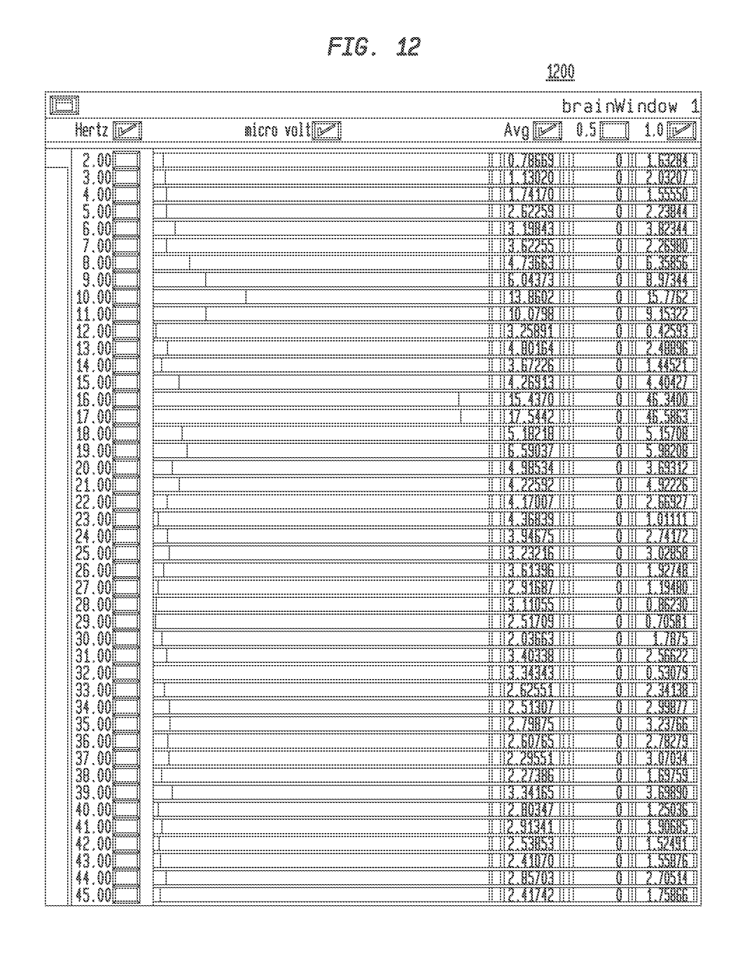

FIG. 12 shows a brain state chart 1200 having a dominance 16 Hz and 17 Hz brainwave activity which is characterized as a directed awareness of the proprioceptive sense of “feeling” a part of the body without reference to warmth, heaviness, position, temperature, or other proprioceptive cues.

FIG. 13 shows a brain state chart 1300 characterizing a proprioceptive sense encompassing feeling of physical warmth and/or directed awareness of skin surface temperature in a specific body area (e.g., warm hands).

Additionally, in the absence of seizure activity or brain damage, the 5 Hz and 6 Hz brainwave bands are characterized as co-varying in a surging manner when the subject experiences self-perceived fatigue. The 15 and 16 Hz brainwaves co-vary in a surging manner when a subject is in touch with how a specific body part or area is “feeling.”

Once the brain states are correlated to the EEG and other biologic data, specifically the pairs of brainwaves, the subject can be taught to change his or her brain state in response to auto-stimulation via a personal cue. In one embodiment, a method of treatment of ADHD can be applied.

### Example 1

Subject is a 43-year old male who had previously been diagnosed with ADHD. He was monitored using the equipment described hereinabove to produce a series of baseline EEG's, brain state charts, and effective cues to voluntarily effect shifts in symptomology. A baseline was established as well as signature scans of various brain states (e.g., mindfulness, clarity of mind, mental fog, racing mind, calm mind, “heaviness” in arms, hands, and fingers, and a sense of surface temperature on arms, hands, and fingers) experienced during a first session. This does not limit the types and kinds of brain and/or proprioceptive states that can be used in the baseline session. Using training screens, the subject learned what brainwave signatures were associated with his consciousness, mindfulness, thoughts and proprioceptive body-sense. The subject chose to work to control his “racing mind” brain state during future sessions. During a second session, when the subject experienced surges in 9 Hz and 10 Hz, showing a “racing mind,” he received auditory feedback of the terms “racing mind” and conscientiously changed his brain state to achieve a preferable brainwave scan in the same manner described with reference to Example 2 below.

While treatment involved multiple sessions, the subject reported significant improvement after the second session (i.e., the number of occurrences of racing mind in the subject was less in the time period after the session as compared to the number of occurrences of racing mind in the subject prior to the session), which was evidenced by a brainwave scan showing an increase in the occurrence of 13 Hz and 14 Hz brainwaves (i.e., indicating calm) and a decrease in the occurrence of the 9 Hz and 10 Hz brainwaves (i.e., indicating racing mind). FIG. 9 shows a scan 900 taken during the session, reflecting what the subject experienced as a “racing mind,” and a scan 1200 shown in FIG. 12 taken at the end of the session and showing a calm mind and release of muscular tension as a change in the brain state of the subject.

### Example 2

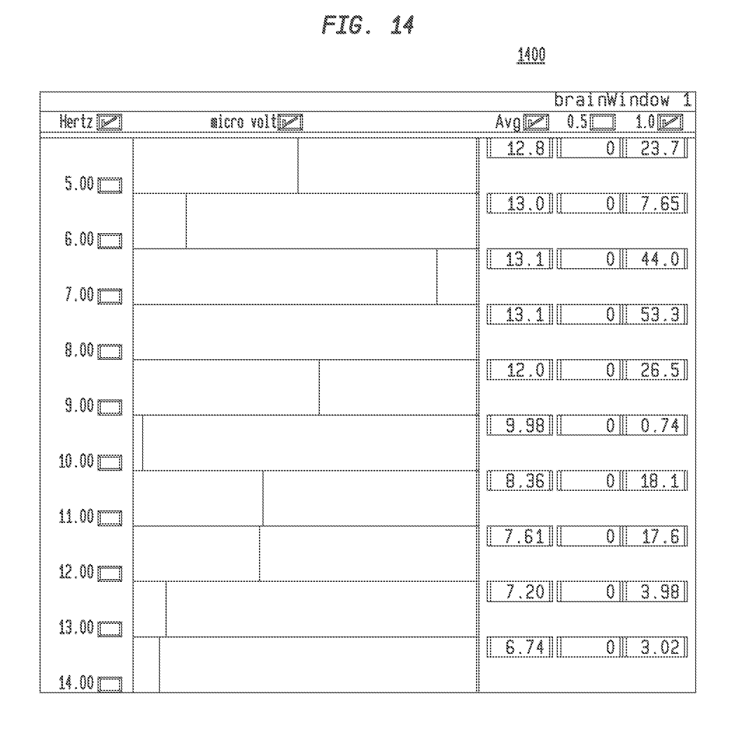

Referring now to FIGS. 14 through 19, depicted are a series of brain state charts that result during a session with a subject such as that described above with respect to process 1300. FIG. 14 depicts a brain chart 1400 associated with the approximately one (1) second first epoch of the onset and/or detection of a predetermined undesirable and/or problematic brain state. In the depicted embodiment, brain chart 1400 includes ten (10) brainwave bands, i.e., one Hertz bands in the range of five (5) Hertz through fourteen (14) Hertz. For the purposes of this example, the onset/detection epoch is epoch one of four. In this exemplary embodiment, the undesirable and/or problematic brain state is intrusive mental imagery as also depicted in brain state chart 800 of FIG. 8. The detected brain state exemplified on the training screen of FIG. 14 shows a clear surge to dominance of 53.3 microvolts and 26.5 microvolts for the 7 Hz and 8 Hz brainwaves, respectively. Of interest, during epoch one, the values of 3.98 microvolts and 3.02 microvolts associated with the 13 Hz and 14 Hz brainwaves, respectively, indicate a lack of, or minimal proprioception of, the brain state associated with a dominance of a sense of heaviness in limbs, hands, and/or digits and/or of muscular tension in the subject's current brain state.

In this example, immediately upon the detection of the undesirable intrusive mental imagery brain state, a cue of “heavy thumb” initiated by the user of the process to the subject of the process. In this example, the cue of heavy thumb is provided by merely stating these words to the subject. The goal of providing this cue is to train the subject to pair the cue to awareness of the occurrence of the undesirable brain state and recognize its occurrence. And, simultaneously or near simultaneously, to redirect the subject's thoughts to change from the intrusive mental imagery dominated brain state to a brain state dominated by muscular calm in a body part (i.e., heavy thumb). This is done by the subject focusing on the weight of his or her thumb.

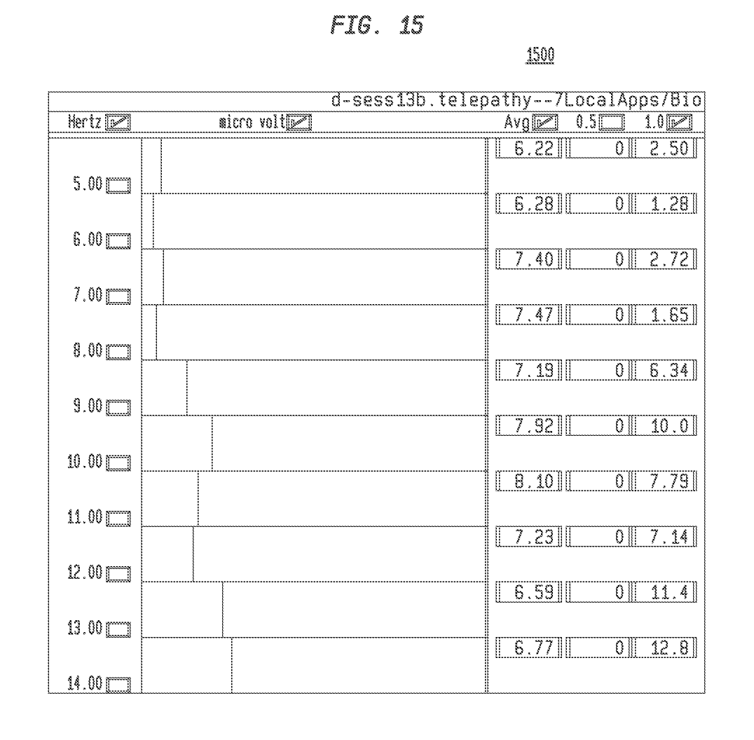

Turning now to FIG. 15, depicted is a brain state chart for epoch two. Epoch two is the brain state that occurs after presentation of a stimulus such as the heavy thumb cue. The duration of epoch two, in this example, is approximately one (1) second. As seen in brain chart 1500, the values associated with the 7 Hz and 8 Hz brainwave pair greatly decrease from the initial 53.3 and 26.5 microvolts, respectively, to 2.72 and 1.65 microvolts, respectively, and the values associated with the 13 Hz and 14 Hz brainwave pair increase from the initial values of 3.98 and 3.02 microvolts, respectively, to 11.4 and 12.8 microvolts, respectively. This data shows a shift in the brain state away from intrusive mental imagery and towards a brain state enabling a proprioception of a specific area of muscular calm.

Upon the occurrence of the brain state shown in FIG. 15, and during the second epoch, the stimulus is repeated. That is, in this example, the words “heavy thumb” are repeated by the user of the process to the subject. Again, the goal of repeating this cue is to reinforce the subject's redirection of the subject's brain state dominated by intrusive thoughts toward a brain state enabling a proprioception of a specific area of muscular calm (i.e., heavy thumb).

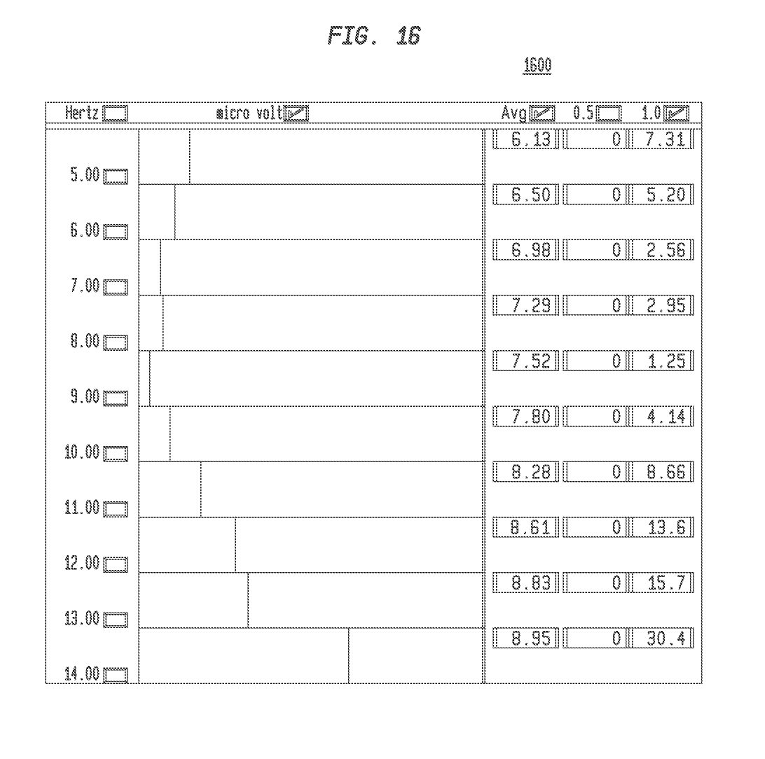

Next, as seen in FIG. 16, depicted is a brain state chart for epoch three. Epoch three is the brain state that occurs after the second presentation of a stimulus such as a heavy thumb cue. The duration of epoch three, in this example, is approximately one (1) second. As seen in brain chart 1600, the values associated with the 7 Hz and 8 Hz brainwave pair stay at a level greatly decreased from the initial 53.3 and 26.5 microvolts, respectively, i.e., 2.56 and 2.95 microvolts, respectively, and the values associated with the 13 Hz and 14 Hz brainwave pair continue to increase from the initial values of 3.98 and 3.02 microvolts, and the secondary values of 11.4 and 12.8 microvolts, respectively, to a new level of 15.7 and 30.4 microvolts, respectively. This data shows a continuing shift in the brain state away from intrusive mental imagery and towards a specific area of muscular calm.

Upon the occurrence of the brain state shown in FIG. 16, and during the third epoch, the stimulus is again repeated. That is, in this example, the words “heavy thumb” are repeated by the user of the process to the subject. Again, the goal of repeating this cue is to reinforce the subject's redirection of the subject's brain state dominated by intrusive thoughts toward a brain state and proprioception dominated by muscular calm in a body part (i.e., heavy thumb).

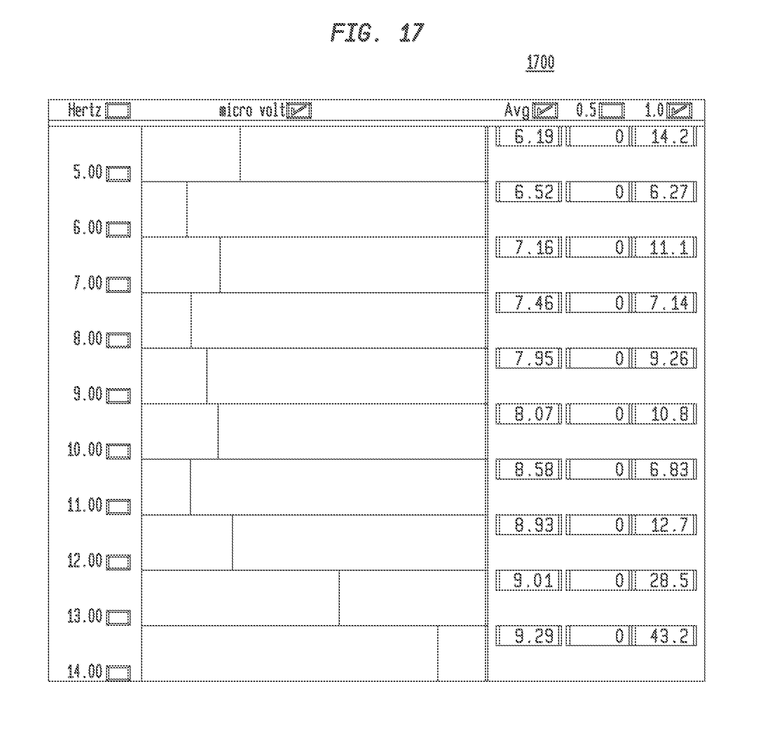

Next, as seen in FIG. 17, depicted is a brain state chart for epoch four. Epoch four is the brain state that occurs after the third presentation of a stimulus such as a heavy thumb cue. The duration of epoch four, in this example, is approximately one (1) second. As seen in brain chart 1700, the values associated with the 7 Hz and 8 Hz brainwave pair stay at a level greatly decreased from the initial 53.3 and 26.5 microvolts, respectively, i.e., 11.1 and 7.14 microvolts, respectively, and the values associated with the 13 Hz and 14 Hz brainwave pair continue to increase from the initial values of 3.98 and 3.02 microvolts, the secondary values of 11.4 and 12.8 microvolts, respectively, the tertiary values of 15.7 and 30.4 microvolts, respectively, to a new level of 28.5 and 43.2 microvolts, respectively. This data shows a continuing shift from a brain state and proprioception dominated by intrusive mental thoughts to a brain state and proprioception dominated by muscular calm in a body part (i.e., heavy thumb). Through this training, the subject can learn to independently identify and alter his or her brain state via, for example, redirection of the intrusive thoughts/imagery toward a more desirable brain state.

### Example 3

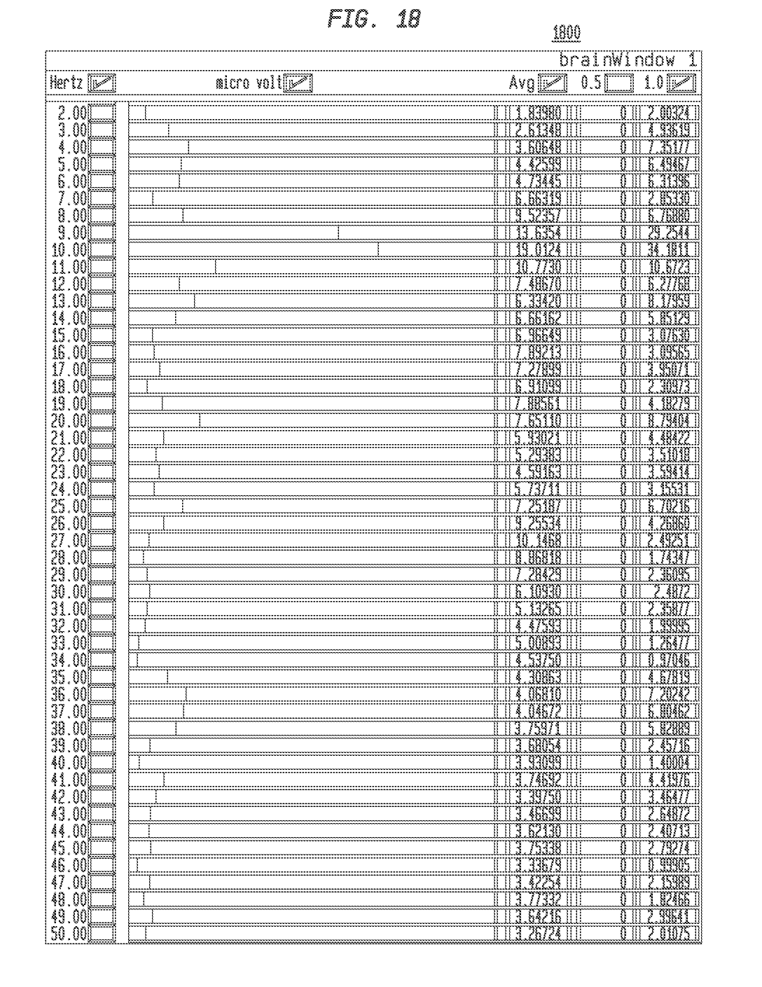

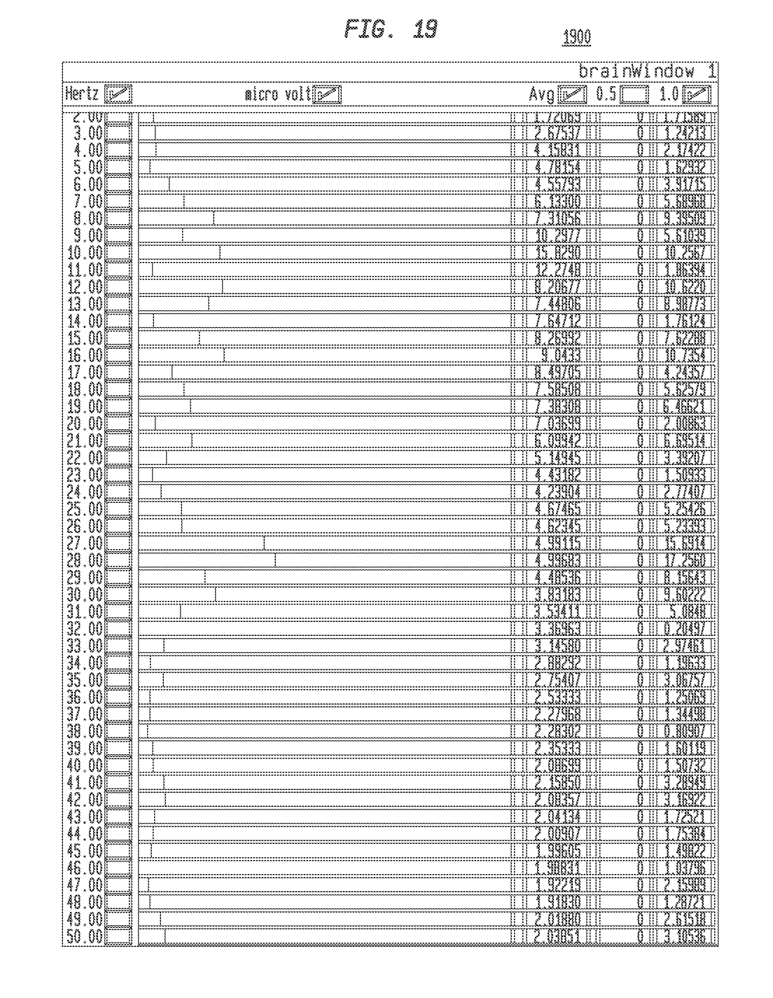

Referring now to FIGS. 18 through 19, depicted are a series of brain state charts that result during a session with a subject such as that described above with respect to process 1300. FIG. 18 depicts a brain chart 1800 associated with the approximately one (1) second first epoch of the onset and/or detection of a predetermined undesirable and/or problematic brain state. In the depicted embodiment, brain chart 1800 includes forty-nine (49) brainwave bands, i.e., one Hertz bands in the range of two (2) Hertz through fifty (50) Hertz. For the purposes of this example, the onset/detection epoch is epoch one of a plurality of epochs. In this exemplary embodiment, the undesirable and/or problematic brain state is racing mind as is often associated with ADHD and as also depicted in brain state chart 1800 of FIG. 18. The detected brain state exemplified on the training screen of FIG. 18 shows a clear surge to dominance of 29.2544 microvolts and 34.1811 microvolts for the 9 Hz and 10 Hz brainwaves, respectively, which is representative of a dominance of a racing mind brain state.

In this example, immediately upon the detection of the undesirable intrusive racing mind brain state, a cue of “calm mind” is initiated by the user of the process to the subject of the process. In this example, the cue of calm mind is provided by merely stating these words to the subject. The goal of providing this cue is to train the subject to identify the undesirable brain state and recognize its occurrence. And, simultaneously or near simultaneously, to redirect the subject's thoughts to change from the racing mind dominated brain state to a brain state dominated by a calm mind. This is done by the subject learning to volitionally shift to a calm mind.

Turning now to FIG. 19, depicted is a brain state chart 1900 depicting an exemplary brain state at the end of a, for example, thirty (30) minute session in which steps such as those described in process 1300 and Example 2 are performed but with the intent of changing a racing mind dominant brain state to a calm mind dominant brain state. That is, this brain state chart represents the brain state that occurs after presentation of a stimulus such as a calm mind cue repeatedly throughout a thirty (30) minute session. As seen in brain chart 1900, the values associated with the 9 Hz and 10 Hz brainwave pair greatly decrease from the initial 29.2544 and 34.1811 microvolts, respectively, to 5.61039 and 10.2567 microvolts, respectively. This data shows a shift in the brain state away from racing mind. In this manner, the subject is trained regarding the awareness of the occurrence of the undesirable brain state (i.e., racing mind) and volitional shifting ability of the subject away from the undesirable brain state.

Although several processes have been disclosed herein as software, it may be appreciated by one of skill in the art that the same processes, functions, etc. may be performed via hardware or a combination of hardware and software. Similarly, although the present invention has been depicted as a hardwired system, these concepts may be applied to wireless systems and hybrid hardwired and wireless systems without departing from the scope of the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for assisting a subject to learn to independently identify and alter a brain state of the subject comprising the steps of:
(i) fitting the subject with equipment to monitor biologic data, the biologic data including a plurality of EEG frequencies in a frequency range;
(ii) requesting the subject retain the eyes in a normal open and blinking position for a second time period;
(iii) monitoring biologic data during the second time period for the occurrence of a predetermined brain state;
(iv) upon the detection of the predetermined brain state, presenting a stimulus to the subject, the stimulus designed to evoke an action performed by the subject, the action intended to alter the brain state;
(v) repeating steps (iii) and (iv).

2. A method according to claim 1 further comprising the step of:
(vi) recording the biologic data during the second time period.

3. A method according to claim 1, wherein the action includes the subject intentionally and voluntarily altering the brain state via altering of a thinking process of the subject.

4. A method according to claim 1, wherein adjacent frequencies in the frequency range are separated by approximately one hertz.

5. A method according to claim 4, wherein the frequency range begins with two hertz and ends with one hundred and ninety hertz.

6. A method according to claim 1, wherein the monitoring biologic data includes monitoring of at least one of four change states or a combination thereof.

7. A method according to claim 6, wherein:
a first change state is an increase in amplitude in excess of a predetermined percentage for any one of the frequencies in the frequency range;
a second change state is a decrease in amplitude in excess of a predetermined percentage for any one of the frequencies in the frequency range;
a third change state is an increase in amplitude in excess of a predetermined percentage for a first one of the frequencies and a simultaneous decrease in amplitude for a second one of the frequencies; and
a fourth change state is a decrease in amplitude in excess of a predetermined percentage for a first one of the frequencies and a simultaneous increase in amplitude for a second one of the frequencies.

8. A method according to claim 1, wherein the predetermined brain state of perceived mental imagery is detected via a simultaneous surge of the frequency of seven hertz and the frequency of eight hertz.

9. A method according to claim 1, wherein the predetermined brain state of perceived racing mind is detected via a simultaneous surge of the frequency of nine hertz and the frequency of ten hertz.

10. A method according to claim 1, wherein the predetermined brain state enabling a proprioceptive perception of increased or decreased muscular tension or heaviness is detected via a simultaneous surge of the frequency of thirteen hertz and the frequency of fourteen hertz.

11. A method according to claim 1, wherein the predetermined brain state enabling a proprioceptive perception of perceived increased or decreased body temperature is detected via a simultaneous surge of the frequency of twenty seven hertz and the frequency of twenty eight hertz.

12. A method according to claim 1, wherein the predetermined brain state enabling a proprioceptive perception of perceived feeling of a part of the body is detected via a simultaneous surge of the frequency of sixteen hertz and the frequency of seventeen hertz.

13. A method according to claim 1 further comprising the steps of:
(vii) requesting the subject retain eyes in a closed position for throughout a first time period; and
(viii) recording the biologic data during the first time period to create a baseline biologic data signature.

14. A method according to claim 1 wherein the predetermined brain state is selected from the group consisting of mindfulness, clarity of mind, mental fog, racing mind, calm mind, heaviness in arms, heaviness in hands, heaviness in fingers, a sense of arm surface temperature, a sense of hand surface temperature, a sense of finger surface temperature, and combinations thereof.

15. A method according to claim 1, wherein the stimulus is a cue administered to the subject.

16. A method according to claim 1, wherein the action is volitional.

17. A method according to claim 10, wherein the predetermined brain state of perceived increased or decreased muscular tension includes a perceived sense of heaviness in at least one of the group consisting of arms, legs, hands, fingers, toes, and combinations thereof.

18. A method according to claim 12, wherein the predetermined brain state of perceived feeling of a part of the body is an awareness of the feeling without reference to at least one of the group consisting of warmth, heaviness, position, temperature, and combinations thereof.
